SEQUENCE LISTING (2 sequences)

SEQ ID NO: 1 - DNA, 30 bp, artificial sequence
  source: /note=“Description of artificial sequence DNA encoding the optimized CPP sequence”
tgtcgttggcgttggaaatgttgtaaaaaa

SEQ ID NO: 2 - protein, 10 aa, artificial sequence
  source: /note=“Description of artificial sequence optimized CPP sequence”
CRWRWKCCKK

(19) United States
(12) Patent Application Publication
(10) Pub. No.: US 2011/0027300 A1
(43) Pub. Date: Feb. 3, 2011

(54) Identification of a novel cysteine-rich cell penetrating peptide

(75) Inventors: Ugurbil Kamil, Tubingen (DE); Jha Deepti, Tubingen (DE); Engelmann Jorn, Tubingen (DE); Mishra Ritu, Tubingen (DE); Wiesmuller Karl-Heinz, Tubingen (DE)

Correspondence Address: DT Ward, PC, 292 Main Street, Suite 2, Groton, MA, 01450, US

(73) Assignee: Max-Planck-Gesellschaft zur Forderung der Wissenschaften e.V., Munchen (DE)

(21) Appl. No.: 12/867,056

(22) PCT Filed: Feb. 13, 2009
(86) PCT No.: PCT/EP2009/001040

§ 371 Date: Oct. 20, 2010

(30) Foreign Application Priority Data
Feb. 13, 2008 (EP) 08002685.9

(51) Int. Cl. A61K 39/00; C07H 21/04; C12N 15/63; C12N 5/10; C12P 21/00; C07K 7/06; C12Q 1/68; A61K 38/08; A61P 35/00; A61P 3/10; A61P 31/00; A61P 29/00; A61P 37/00

(52) U.S. Cl. 424/185.1; 536/23.1; 435/320.1; 435/325; 435/69.1; 530/328; 435/6; 514/21.6; 514/19.3; 514/6.9; 514/2.3

(57) ABSTRACT

The present invention relates to a nucleic acid molecule encoding a peptide capable of being internalized into a cell, wherein said nucleic acid molecule consists of (a) a nucleic acid molecule encoding a peptide having the amino acid sequence of SEQ ID NO: 2; (b) a nucleic acid molecule having the DNA sequence of SEQ ID NO: 1, wherein T is U if the nucleic acid molecule is RNA; or (d) a nucleic acid molecule encoding a peptide having at least 80% sequence identity with that of SEQ ID NO: 2, wherein at least at two positions selected from the group consisting of positions 1, 7 and 8 of SEQ ID NO: 2 a cysteine is present and wherein at least at four positions selected from the groups consisting of position 2, 4, 6, 9 or 10 of SEQ ID NO: 2 an arginine or a lysine is present. The present invention also relates to a peptide encoded by the nucleic acid of the invention, a fusion molecule comprising the peptide of the invention and a composition comprising the peptide or the fusion molecule of the invention. Furthermore, the present invention relates to a method of detecting the internalization behaviour of a fusion molecule of the invention, the composition of the invention for treating and/or preventing a condition selected from cancer, enzyme deficiency diseases, infarcts, cerebral ischemia, diabetes, inflammatory diseases, infections such as bacterial, viral or fungal infections, autoimmune diseases such as systemic lupus erythematodes (SLE) or rheumatoid arthritis, diseases with amyloid-like fibrils such as Alzheimer's disease (AD) and Parkinson's disease (PD) or certain forms of myopathy.

The present invention relates to a nucleic acid molecule encoding a peptide capable of being internalized into a cell, wherein said nucleic acid molecule consists of (a) a nucleic acid molecule encoding a peptide having the amino acid sequence of SEQ ID NO: 2; (b) a nucleic acid molecule having the DNA sequence of SEQ ID NO: 1, wherein T is U if the nucleic acid molecule is RNA; or (c) a nucleic acid molecule encoding a peptide having at least 80% sequence identity with that of SEQ ID NO: 2, wherein at least at two positions selected from the group consisting of positions 1, 7 and 8 of SEQ ID NO: 2 a cysteine is present and wherein at least at four positions selected from the group consisting of positions 2, 4, 6, 9 or 10 of SEQ ID NO: 2 an arginine or a lysine is present. The present invention also relates to a peptide encoded by the nucleic acid of the invention, a fusion molecule comprising the peptide of the invention and a composition comprising the peptide or the fusion molecule of the invention. Furthermore, the present invention relates to a method of detecting the internalization behaviour of a fusion molecule of the invention, the composition of the invention for treating and/or preventing a condition selected from cancer, enzyme deficiency diseases, infarcts, cerebral ischemia, diabetes, inflammatory diseases, infections such as bacterial, viral or fungal infections, autoimmune diseases such as systemic lupus erythematodes (SLE) or rheumatoid arthritis, diseases with amyloid-like fibrils such as Alzheimer's disease (AD) and Parkinson's disease (PD) or certain forms of myopathy.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference, to the same extent as if each individual document was specifically and individually indicated to be incorporated by reference.

The targeted delivery of substances to cells has long been hampered by the cell membrane being an efficient protective wall to exclude most molecules that are not actively imported by living cells. Only a narrow range of molecules of certain molecular weight, polarity and net charge is able to diffuse through cell membranes. Other molecules have to be actively transported by e.g. receptor-mediated endocytosis or artificially forced through the cell membrane by methods such as electroporation, cationic lipids/liposomes, micro-injection, viral delivery or encapsulation in polymers. These methods are mainly utilized to deliver hydrophobic molecules. Furthermore, the side effects associated with these methods and the fact that their utilization is limited to in vitro uses has prevented them from becoming an efficient means to deliver substances such as drugs to the cell in order to treat diseases and conditions.

The discovery of cell-penetrating peptides (CPPs) also called protein transduction domains (PTDs) or membrane translocation sequences (MTS) proved that the translocation of larger molecules through the cell membrane is possible. Prominent examples of CPPs are the HIV-1 TAT translocation domain (Green and Loewenstein, 1988) and the homeodomain of the Antennapedia protein from Drosophila (Joliot et al., 1991). The exact translocation mechanism is still disputed. Mutation studies of the Antennapedia protein revealed that a sequence of 16 amino acids called penetratin or pAntp (Derossi et al., 1994) is necessary and sufficient for membrane translocation. In the following, other protein-derived CPPs were developed such as the basic sequence of the HIV-1 Tat protein (Vivès et al., 1997) and the chimeric peptide transportan (Pooga et al., 1998). A synthetic peptide developed is the amphipathic model peptide (Oehlke et al., 1998). Coupling of antisense DNA or PNAs to CPPs was shown to exert the desired effect in vivo.

It was long questioned which features were necessary for a CPP to exert the translocation function. In general, little structural resemblance has been found between the different families of CPPs. So far the only consistently found feature is the high content of basic amino acids resulting in a positive net charge. Thus, it is assumed that CPPs initially bind to negatively charged head groups of lipids or proteins in the cell membrane. In this regard, the importance of arginine as positive amino acid was demonstrated by several groups (Rothbard et al., 2000; Wender et al., 2000). Generally, an alpha-helical secondary structure has been predicted for CPPs which could be verified for some cases but cannot be taken as a general prerequisite.

Many proteins able to translocate have severe side-effects on the cell, which is understandable in view of the fact that most of the naturally occurring substances are used as e.g. antimicrobial substances or toxins. CPPs can e.g. cause cytoplasmic leakage due to membrane disruption and also interfere with the functioning of membrane proteins. CPPs might also exhibit cellular toxic effects, such as e.g. transportan which affects GTPase activity (Soomets et al., 2000). Furthermore, it becomes more and more clear that many CPPs only exert their function under certain very narrow conditions which cannot be met in vivo. Another drawback is that, depending on the target cell, the CPPs may be rapidly degraded in the cells. Lastly, toxic and immunogenic effects of CPPs have been observed which prevent their utilization e.g. in therapeutic applications.

Up to now and depending on the mechanism of internalization, known CPPs mainly localize in the nucleus or, in case they are internalized in vesicles, remain there and only a small part is released into the cytoplasm.

Crotamine is one of the main toxins in the venom of the South American rattlesnake (Rádis-Baptista et al., 1999) and shows high homology with other venom myotoxins. The 42 amino acid long cationic polypeptide contains 11 basic residues and six cysteines giving rise to three disulfide bonds. It has two putative NLS motifs, Crot₂₋₁₈ and Crot₂₇₋₃₉. Crotamine was shown to be a CPP penetrating into different cell types and mouse blastocysts in vitro (Kerkis et al., 2004). It was shown to be non-toxic to a concentration of up to 1 μM and to localize preferably in the nucleus where it is supposed to bind to chromatin structures. When applied before cell division, crotamine is mainly localized in the cytoplasm after the telophase. The two peptides corresponding to the putative NLS motifs were examined in WO2006/096953, where it was found that both are able to internalize into cells. It was further concluded from the experiments conducted that the peptide corresponding to Cro₂₋₁₈ was able to more efficiently internalize and transport DNA encoding GFP into cells than Cro₂₇₋₃₉. The present inventors surprisingly found that it is possible to provide a CPP which is even shorter than the minimum sequence of the NLS motif Cro₂₇₋₃₉ determined in Kerkis et al. (2004) and examined in WO2006/096953.

Accordingly, the present invention relates to a nucleic acid molecule encoding a peptide capable of being internalized into a cell, wherein said nucleic acid molecule consists of (a) a nucleic acid molecule encoding a peptide having the amino acid sequence of SEQ ID NO: 2; (b) a nucleic acid molecule having the DNA sequence of SEQ ID NO: 1, wherein T is U if the nucleic acid molecule is RNA; or (c) a nucleic acid molecule encoding a peptide having at least 80% sequence identity with that of SEQ ID NO: 2, wherein at least at two positions selected from the group consisting of positions 1, 7 and 8 of SEQ ID NO: 2 a cysteine is present and wherein at least at four positions selected from the group consisting of positions 2, 4, 6, 9 or 10 of SEQ ID NO: 2 an arginine or a lysine is present.

The term “nucleic acid molecule” as used interchangeably with the term “polynucleotide”, in accordance with the present invention, includes DNA, such as cDNA or genomic DNA, and RNA. If the nucleic acid molecule is RNA, thymine (T) bases denoted in e.g. SEQ ID NO: 1 are replaced with uracil (U), the thymine analogue occurring in RNA. Further included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA) and locked nucleic acid (LNA) (see Braasch and Corey, Chem Biol 2001, 8: 1). LNA is an RNA derivative in which the ribose ring is constrained by a methylene linkage between the 2′-oxygen and the 4′-carbon. They may contain additional non-natural or derivative nucleotide bases, as will be readily appreciated by those skilled in the art. For the purposes of the present invention, also a peptide nucleic acid (PNA) can be applied. Peptide nucleic acids have a backbone composed of repeating N-(2-aminoethyl)-glycine units linked by peptide bonds. The purine and pyrimidine bases are linked to the backbone by methylene carbonyl bonds.

In a preferred embodiment, the nucleic acid molecule is DNA.

The term “peptide” as used herein describes linear molecular chains of amino acids, including fragments of single chain proteins, containing up to 30 amino acids. Peptides may form oligomers consisting of at least two identical or different molecules. The corresponding higher order structures of such multimers are, correspondingly, termed homo- or heterodimers, homo- or heterotrimers etc. The term “peptide” furthermore comprises peptidomimetics of such peptides where amino acid(s) and/or peptide bond(s) have been replaced by functional analogues. Such functional analogues also include all known amino acids other than the 20 gene-encoded amino acids, such as selenocysteine. In principle, it is possible that the peptide of up to 30 amino acids consists only of one or several copies of the peptide of the invention. Alternatively, the peptide may be fused to a second peptide that does not naturally occur in conjunction with the peptide of the invention and is preferably heterologous thereto.

A polypeptide as used in the context of the present invention contains more than 30 amino acids. In accordance with the invention, the term is interchangeably used with “protein” and applies in cases where the peptide of the invention is either multimerized or fused to another peptide or polypeptide to form a fusion molecule according to the invention, as will be described further below.

The term “capable of being internalized” as used in the context of the present invention refers to the ability of some peptides to pass the plasma membrane of cells or to direct the passage of fusion molecules comprising said peptides through the plasma membrane of cells. Different mechanisms of internalization are proposed in the literature: an energy-dependent endocytotic mechanism and an energy-independent passive transport mechanism. The latter can be further divided into several suggested models. In the inverted micelle-driven delivery model, the positively charged part of the CPP interacts with the phospholipids in the membrane, followed by the interaction of the hydrophobic part of the peptide with the membrane, creating the inverted micelle. Another model suggests direct penetration of the plasma membrane. It was suggested by example of the TAT peptide that the mechanism of translocation depends on the cargo attached/fused to the peptide. Size may play a role as well as the chemical properties of the cargo. Furthermore, it was shown that the mechanisms may vary depending on the concentration of CPP. For a recent review see e.g. Tréhin and Merkle (2004), Magzoub and Gräslund (2004) or Gupta et al. (2005). In the context of the present invention, any possible mechanism of internalization is envisaged. A preferred mechanism would ensure that at least a part, preferably more than 30%, more preferably more than 40%, even more preferably more than 50%, even more preferably more than 60%, even more preferably more than 70% and most preferably more than 80% of the CPP or a CPP conjugate/fusion localizes in the cytoplasm in contrast to localization in different compartments, e.g. in vesicles, endosomes or in the nucleus.

As regards the presence of specific amino acids at certain positions of the peptide encoded by the nucleic acid molecule of the present invention, these positions can also be assigned to the sequence of said peptide if it is present in a longer peptide or protein. More particularly, if the stretch of amino acids homologous or identical to the peptide corresponding to SEQ ID NO: 2 or encoded by SEQ ID NO: 1 is identified in (a nucleic acid sequence encoding) a longer peptide or protein, both sequences can be aligned and the positions are assigned. From this information, the positions in the longer peptide or protein corresponding to the respective amino acid in the peptide encoded by SEQ ID NO: 1 can be retrieved.

In accordance with the present invention, the term “percent (%) sequence identity” describes the number of matches (“hits”) of identical amino acids of two or more aligned amino acid sequences as compared to the number of amino acid residues making up the overall length of the template amino acid sequences. In other terms, using an alignment, for two or more sequences or subsequences the percentage of amino acid residues that are the same (e.g., 70%, 80% or 85% identity) may be determined, when the (sub)sequences are compared and aligned for maximum correspondence over a window of comparison, or over a designated region as measured using a sequence comparison algorithm as known in the art, or when manually aligned and visually inspected. This definition also applies to the complement of a test sequence.

To evaluate the identity level between two protein sequences, they can be aligned electronically using suitable computer programs known in the art. Such programs comprise BLAST (Altschul et al., J. Mol. Biol. 1990, 215: 403), variants thereof such as WU-BLAST (Altschul & Gish, Methods Enzymol. 1996, 266: 460), FASTA (Pearson & Lipman, Proc. Natl. Acad. Sci. USA 1988, 85: 2444) or implementations of the Smith-Waterman algorithm (SSEARCH, Smith & Waterman, J. Mol. Biol. 1981, 147: 195). These programs, in addition to providing a pairwise sequence alignment, also report the sequence identity level (usually in percent identity) and the probability for the occurrence of the alignment by chance (P-value). For amino acid sequences, the BLASTP program uses as default a word length (W) of 3, and an expectation (E) of 10. The BLOSUM62 scoring matrix (Henikoff, Proc. Natl. Acad. Sci., 1992, 89:10915) uses alignments (B) of 50, expectation (E) of 10, M=5, N=4, and a comparison of both strands. Programs such as CLUSTALW (Thompson Nucl. Acids Res. 2 (1994), 4673-4680) can be used to align more than two sequences. In addition, CLUSTALW, unlike e.g. FASTDB, does take sequence gaps into account in its identity calculations.

All of the above programs can be used in accordance with the invention.

In a preferred embodiment of the present invention, the sequence identity to SEQ ID NO: 2 is at least 90% and more preferably at least 95%. It is particularly preferred that the sequence identity to SEQ ID NO: 2 is 100%.

Substitutions in the amino acid sequence of the peptide of the present invention are preferably conservative. This means that substitutions preferably take place within one class of amino acids. For example, a positively charged amino acid is preferably mutated to another positively charged amino acid. The same holds true for the classes of basic, aromatic or aliphatic amino acids.

In the course of the present invention, it has surprisingly been found that a peptide derived from crotamine but having a reduced length as compared to a fragment of crotamine consisting of amino acids 27 to 39 (Cro₂₇₋₃₉) and retaining all cysteines has improved internalization properties as compared to naturally occurring crotamine and comparable or even better internalization properties than Cro₂₇₋₃₉. In comparing the peptide of the invention with other CPPs, the C-terminal nuclear localization signal Cro₂₇₋₃₉ (KMDCRWRWKCCKK) proposed as one of two potential sequences responsible for membrane permeation (Kerkis et al., 2004) was separately examined for its internalization properties. The peptide corresponding to Cro₂₇₋₃₉ was chosen for further investigation despite the disclosure of WO2006/096953 proposing the peptide corresponding to the N-terminal nuclear localization signal Cro₂₋₁₈ as a more promising CPP than Cro₂₇₋₃₉. The uptake behavior of this fragment Cro₂₇₋₃₉ was studied with a fluorophore attached to it. It proved to be an efficient CPP also showing cytoplasmic diffusion. Cytoplasmic diffusion is particularly desirable to deliver pharmacologically active substances to the cell. This additional feature led the present inventors to introduce changes to this fragment to avoid the use of amino acids like methionine, tryptophan, aspartic acid and, in particular, cysteine which makes the synthesis more complex and challenging. Avoiding cysteines will not only facilitate the synthesis, handling and storage of the peptide but is also suggested to improve the in vivo properties of the peptide since cysteines will likely form intra- and intermolecular cysteine bridges and therefore promote aggregation of the peptide. However, up to now, the role of cysteines or cysteine bridges in CPP has not been examined. International patent application WO03/106491 discloses a method for predicting or designing CPPs. It is of note that the majority of peptides predicted and/or shown to exert CPP properties do not contain any cysteine.

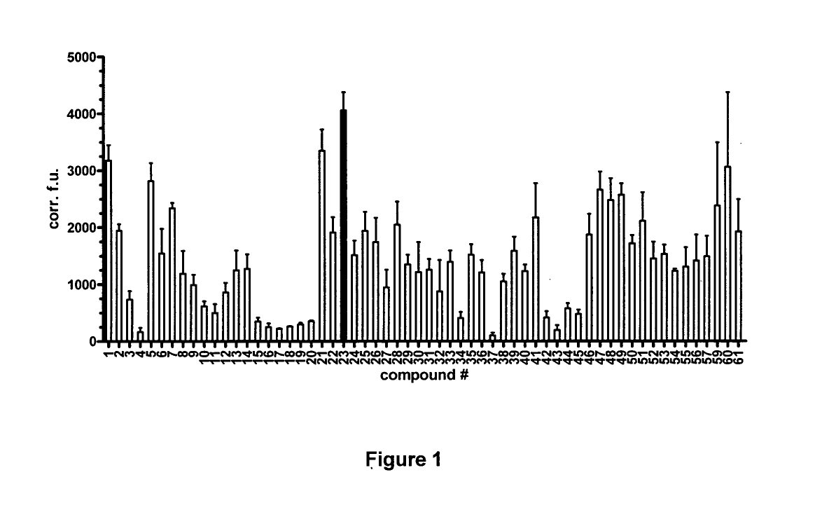

Positively charged amino acids (lysine and arginines) were not modified as these are an important feature of a cell penetrating peptide. Different derivatives wherein cysteines were substituted with alpha amino butyric acid or serine (close analogues of cysteine) were synthesized as well as fragments of the sequence Cro₂₇₋₃₉ by deleting amino acids from the N-terminus. Alternatively, cysteines were deleted one by one or amino acids anywhere in the sequence were deleted. Finally, tryptophan was substituted by proline or phenylalanine. All combinations were synthesized containing a lysine at the N-terminus as a linker for the coupling of FITC (at the ε-amino group) for analyzing samples for intracellular uptake by fluorescence imaging. The internalization studies on cultured cells show that a change in the amino acid composition dramatically affects the intracellular uptake (see FIGS. 1 to 3 and the examples). Not only the deletion of cysteines one by one in different combinations but also the substitution by α-aminobutyric acid reduced the cellular uptake with the number of cysteines deleted or substituted. Substitution of cysteines by serine shows nearly the same results. Since proline-rich peptides are known to enhance cell permeability, tryptophan was substituted with proline. However, internalization was again immensely affected to the negative. The best results are observed for a fragment K(FITC)-CRWRWKCCKK (peptide 23 of table 1 below displaying a complete list of all sequences examined for their internalization properties) which is competently taken up by cells and also show endosomal as well as cytoplasmic fluorescence distribution. This fragment without the linker is three amino acids shorter than the original fragment Cro₂₇₋₃₉ having the sequence KMDCRWRWKCCKK) keeping the number of the cysteines the same. Studies carried out on well known CPPs like Tat, Antennapedia and polyarginines revealed that the role of the positive charge is crucial for translocation. Unlike the known CPPs the CPP of the present invention is markedly different in terms of its function. Efficient cellular uptake and cytosolic location along with vesicular distribution at subtoxic concentrations (<2.5 μM) is the distinguishing feature which is also shown by other CPP but at comparatively high concentrations (>10 μM). Other distinctive features are the influence of the chirality of the peptide backbone as well as the sequence order on the cellular uptake: Forms of the proposed peptide with the sequence of the CPP of the present invention reversed, the sequence of the CPP of the present invention with D-amino acids, or with D-amino acids and in reversed order also showed lower uptake and cytosolic diffusion unlike known for the Tat peptide.

The entire study shows the significance of each amino acid focusing the requirement of charge and hydrophobic residues during membrane permeation. As known from previous studies charged residues help to adhere to the cell surface which is the first step of internalization and then tryptophan might aid membrane translocation by membrane destabilization.

TABLE 1
Sequences derived from Cro₂₇₋₃₉ and examined
for their internalization properties in
the present invention.

| 1 | K (FITC)-KMDCRWRWKCCKK (Cro₂₇₋₃₉) |
|---|---|
| 2 | K (FITC)-KMDXRWRWKCCKK |
| 3 | K (FITC)-KMDXRWRWKXCKK |
| 4 | K (FITC)-KMDXRWRWKXXKK |
| 5 | K (FITC)-KMDXRWRWKCXKK |
| 6 | K (FITC)-KMDCRWRWKCXKK |
| 7 | K (FITC)-KMDCRWRWKCSKK |
| 8 | K (FITC)-KMDCRWRWKSCKK |
| 9 | K (FITC)-KMDSRWRWKCCKK |
| 10 | K (FITC)-KMDCRWRWKSSKK |
| 11 | K (FITC)-KMDSRWRWKSSKK |
| 12 | K (FITC)-KMDSRWRWKSCKK |
| 13 | K (FITC)-KMDSRWRWKCSKK |
| 14 | K (FITC)-KMDCRWRPKCCKK |
| 15 | K (FITC)-KMDCRPRPKCCKK |
| 16 | K (FITC)-KMDXRPRPKCCKK |
| 17 | K (FITC)-KMDXRPRPKXCKK |
| 18 | K (FITC)-KMDXRPRPKCXKK |
| 19 | K (FITC)-KMDCRPRPKXCKK |
| 20 | K (FITC)-KMDCRPRPKCXKK |
| 21 | K (FITC)-MDCRWRWKCCKK |
| 22 | K (FITC)-DCRWRWKCCKK |
| 23 | K (FITC)-CRWRWKCCKK [K(FITC)-CyLoP-1] |
| 24 | K (FITC)-RWRWKCCKK |
| 25 | K (FITC)-MDCRWRWKXCKK |
| 26 | K (FITC)-DCRWRWKXCKK |
| 27 | K (FITC)-DCRWRWKCXKK |
| 28 | K (FITC)-CRWRWKXCKK |
| 29 | K (FITC)-CRWRWKCXKK |
| 30 | K (FITC)-RWRWKXCKK |
| 31 | K (FITC)-MDCRWRWKXXKK |
| 32 | K (FITC)-DCRWRWKXXKK |
| 33 | K (FITC)-CRWRWKXXKK |
| 34 | K (FITC)-RWRWKXXKK |
| 35 | K (FITC)-KMDCRWRWKCKK |
| 36 | K (FITC)-KMDCRWRWKKK |
| 37 | K (FITC)-KMDRWRWKKK |
| 38 | K (FITC)-KDCRWRWKCCKK |
| 39 | K (FITC)-KCRWRWKCCKK |
| 40 | K (FITC)-KRWRWKCCKK |
| 41 | K (FITC)-CRWRWKCSKK |
| 42 | K (FITC)-CRWRWKSSKK |
| 43 | K (FITC)-SRWRWKSSKK |
| 44 | K (FITC)-SRWRWKCSKK |
| 45 | K (FITC)-SRWRWKSCKK |
| 46 | K (FITC)-SRWRWKCCKK |
| 47 | K (FITC)-CRFRWKCCKK |
| 48 | K (FITC)-CRWRFKCCKK |
| 49 | K (FITC)-CRFRFKCCKK |
| 50 | K (FITC)-KCCKWRWRCK |
| 51 | k (FITC)-crwrwkcckk |
| 52 | k (FITC)-kcckwrwrck |
| 53 | K (FITC)-CrWRWKCCKK |
| 54 | K (FITC)-CRwRWKCCKK |
| 55 | K (FITC)-CRWrWKCCKK |
| 56 | K (FITC)-CRWRwKCCKK |
| 57 | K (FITC)-CrwrwKCCKK |
| 58 | K (FITC)-CRWRWKCGCKK |
| 59 | K (FITC)-CCRWRWKCCKK |
| 60 | K (FITC)-CGCRWRWKCGCKK |
| 61 | K (FITC)-CRWRWKCG |

The individual sequences are discussed in further detail in the appended examples.
X: a-aminobutyric acid; Capitals denote L-amino acids, minuscules denote
D-amino acids. Peptide 23 is also denoted as CyLoP: Cytosol Localizing Peptide.

Thus, it has been found that the CPP of the invention actually requires tryptophanes and, surprisingly, cysteines in order to optimally exert its internalization properties. Accordingly, an optimized peptide according to the present invention is encoded by the nucleic acid sequence depicted in SEQ ID NO: 1 or has the amino acid sequence of SEQ ID NO: 2. It encodes three cysteines and two tryptophanes which unexpectedly are shown to improve the internalization properties of the peptide. Being rich in amino acids like methionine, aspartic acid, and cysteine Cro₂₇₋₃₉ endures difficulties during synthesis and storage. Thus it would be desirable to avoid such amino acids as much as possible which is the main goal of this study. Herein we show the development of the proposed fragment from the known one. Both sequences are comparable in terms of function (internalization efficiency and cytosolic distribution) but the proposed sequence seems to give the same results being smaller in length and avoiding at least methionine and aspartic acid.

This result is surprising in view of the fact that, firstly, it was not credible that a peptide having the internalization properties of Cro₂₇₋₃₉ could be further shortened without loss of activity. Secondly, it is unexpected that potentially problematic amino acids such as cysteines are required in the peptide of the invention to exert its internalizing activity. Finally, WO2006/096953 suggests that the peptide corresponding to Cro₂₋₁₈ would be more useful as CPP.

In a preferred embodiment, the peptide encoded by the nucleic acid molecule further comprises a linker.

A linker as used in connection with the present invention is used to connect the peptide of the invention with other moieties. The linker serves to physically separate the peptide of the invention and the other moiety or moieties and to ensure that neither the peptide of the invention nor the other moieties are limited in their function due to the close vicinity to the other. Depending on the other moiety, the linker can be a peptide bond, an amino acid, a peptide of appropriate length, or a different molecule providing the desired features. The skilled person knows how to design appropriate linker molecules, in particular linker peptides based on his common knowledge. For example, peptide linkers can be chosen from the LIP (Loops in Proteins) database (Michalsky et al., 2003). A linker may be appended to the N- or the C-terminus or, if deemed suitable, also to an amino acid apart from the terminal amino acids of the peptide of the present invention. The linker is preferably located at the N-terminus.

A moiety as used in connection with the present invention is a functional unit. A moiety can e.g. be a linker minimally comprising a lysine. Other moieties can be e.g. selectable markers such as FITC or drugs as described in more detail below.

In a more preferred embodiment, the linker is a lysine. The ε-amino group in lysine is suitable to couple the peptide of the invention to various other moieties. Furthermore, the lysine may serve to further improve the internalization properties of the peptide of the invention and may then be supplemented by another linker if deemed suitable.

The present invention also relates to a vector comprising the nucleic acid molecule of the present invention. Preferably, the vector is a plasmid, cosmid, virus, bacteriophage or another vector used conventionally e.g. in genetic engineering.

An expression vector according to this invention is capable of directing the replication and the expression of the nucleic acid molecule of the invention and the peptide or polypeptide encoded thereby. Generally, vectors can contain one or more origins of replication (ori) and inheritance systems for cloning or expression, one or more markers for selection in the host and one or more expression cassettes. Methods which are well known to those skilled in the art can be used to construct and modify recombinant vectors; see, for example, the techniques described in Sambrook and Russell, 2001 and Ausubel, 2001.

The coding sequences inserted in the vector can e.g. be synthesized by standard methods, or isolated from recombinant sources. Ligation of the coding sequences to transcriptional regulatory elements and/or to other amino acid encoding sequences can be carried out using established methods. Transcriptional regulatory elements (parts of an expression cassette) ensuring expression in prokaryotes or eukaryotic cells are well known to those skilled in the art. These elements comprise regulatory sequences ensuring the initiation of the transcription (e.g., translation initiation codon, promoters, enhancers, and/or insulators), internal ribosomal entry sites (IRES) (Owens et al., 2001) and optionally poly-A signals ensuring termination of transcription and stabilization of the transcript. Additional regulatory elements may include transcriptional as well as translational enhancers, and/or naturally-associated or heterologous promoter regions. Preferably, the nucleic acid molecule of the invention is operably linked to such expression control sequences allowing expression in prokaryotes or eukaryotic cells e.g. in the form of a vector. The vector may further comprise nucleotide sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the nucleic acid molecule of the invention. Such leader sequences are well known in the art. Specifically-designed vectors allow the shuttling of DNA between different hosts, such as between bacteria and fungal cells or bacteria and animal cells.

The nucleic acid molecules of the invention as described herein above may be designed for direct introduction or for introduction via liposomes, phage vectors or viral vectors (e.g. adenoviral, retroviral) into the cell. Additionally, baculoviral systems or systems based on Vaccinia Virus or Semliki Forest Virus can be used as vector in eukaryotic expression system for the nucleic acid molecules of the invention. Other expression vectors derived from viruses and usable for delivery of the polynucleotides or vector into targeted cell populations are retroviruses, vaccinia virus, adeno-associated virus, herpes viruses, or bovine papilloma virus.

A typical origin of replication in mammalian vectors is the SV40 viral on. Additional elements in mammalian vectors might include enhancers, Kozak sequences and intervening sequences flanked by donor and acceptor sites for RNA splicing. Highly efficient transcription can be achieved with the early and late promoters from SV40, the long terminal repeats (LTRs) from retroviruses, e.g., RSV, HTLVI, HIVI, and the early promoter of the cytomegalovirus (CMV). However, cellular elements can also be used (e.g., the human actin promoter). Other examples for regulatory elements permitting expression in eukaryotic host cells are the AOX1 or GAL1 promoter in yeast or the CMV-(Cytomegalovirus), SV40-, RSV-promoter (Rous sarcoma virus), chicken beta-actin promoter, CAG-promoter (a combination of chicken beta-actin promoter and cytomegalovirus immediate-early enhancer), the gai10 promoter, human elongation factor 1α-promoter, CMV enhancer, CaM-kinase promoter, the Autographa californica multiple nuclear polyhedrosis virus (AcMNPV) polyhedral promoter or a globin intron in mammalian and other animal cells. Besides elements which are responsible for the initiation of transcription such regulatory elements may also comprise transcription termination signals, such as the SV40-poly-A site or the tk-poly-A site or the SV40, lacZ and AcMNPV polyhedral polyadenylation signals, downstream of the polynucleotide. Suitable selectable markers are dhfr, gpt, G418 neomycin, hygromycin allows the identification and isolation of the transfected cells. The transfected nucleic acid can also be amplified to express large amounts of the encoded peptide. The dhfr (dihydrofolate reductase) marker is useful to develop cell lines that carry several hundred or even several thousand copies of the gene of interest. Another useful selection marker is the enzyme glutamine synthase (GS) (Murphy et al. 1991; Bebbington et al. 1992). Using these markers, the mammalian cells are grown in selective medium and the cells with the highest resistance are selected.

Suitable origins of replication for prokaryotic cells include, for example, the Col E1 and the M 13 origins of replication. Examples of suitable markers for culturing in E. coli and other prokaryotes include tetracycline, kanamycin or ampicillin resistance genes. Possible regulatory elements permitting expression in prokaryotic host cells comprise, e.g., the lac, trp or tac promoter, the lacUV5 or the trp promotor in E. coli.

The nucleic acid molecule of the present invention may be inserted into several commercially available vectors well known to the skilled person who is also able to determine which vectors are suitable for the introduction and/or expression of the peptide of the invention. Non-limiting examples include prokaryotic plasmid vectors, such as the pUC-series, pBluescript (Stratagene), the pET-series of expression vectors (Novagen) or pCRTOPO (Invitrogen), lambda gt11, pJOE, the pBBR1-MCS series, pJB861, pBSMuL, pBC2, pUCPKS, pTACT1 and vectors compatible with expression in mammalian cells like pREP (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, Okayama-Berg cDNA expression vector pcDV1 (Pharmacia), pRc/CMV, pcDNA1, pcDNA3 (Invitrogene), pSPORT1 (GIBCO BRL), pGEMHE (Promega), pLXIN, pSIR (Clontech), pIRES-EGFP (Clontech), pEAK-10 (Edge Biosystems) pTriEx-Hygro (Novagen) and pCINeo (Promega). Examples for plasmid vectors suitable for Pichia pastoris comprise e.g. the plasmids pAO815, pPIC9K and pPIC3.5K (all Invitrogen).

The nucleic acid molecule of the present invention referred to above may also be inserted into vectors such that a translational fusion with another nucleic acid molecule is generated. The other nucleic acid molecule may encode a peptide or protein which may e.g. increase the solubility and/or facilitate the purification of the peptide encoded by the nucleic acid molecule of the invention. Non-limiting examples of such vectors include pET32, pET41, pET43. The additional expressible polynucleotide may also encode one or more chaperones to facilitate correct protein folding. Furthermore, the translational fusion described above may encode a fusion molecule of the invention as will be described elsewhere in this specification.

In another embodiment, the present invention relates to a non-human host transfected or transformed with the vector of the invention.

Non-human hosts according to the invention can be single cells or multi-cellular organisms.

In a more preferred embodiment, the non-human host is a cell.

Suitable prokaryotic host cells comprise e.g. bacteria of the species Escherichia, such as strains derived from BL21 (e.g. BL21(DE3), BL21(DE3)PlysS, BL21(DE3)RIL, BL21(DE3)PRARE) or Rosetta®, Streptomyces, Salmonella or Bacillus. Suitable eukaryotic host cells are e.g. yeasts such as Saccharomyces cerevisiae or Pichia pastoris or insect cells such as Drosophila S2 or Spodoptera Sf9 cells.

Mammalian host cells that could be used include human Hela, HEK293, H9 and Jurkat cells, mouse NIH3T3 and C127 cells, COS1, COS 7 and CV1, quail QC1-3 cells, mouse L cells, Bowes melanoma cells and Chinese hamster ovary (CHO) cells. Also within the scope of the present invention are primary mammalian cells or cell lines. Primary cells are cells which are directly obtained from an organism. Suitable primary cells are, for example, mouse embryonic fibroblasts (MEF), mouse primary hepatocytes, cardiomyocytes and neuronal cells as well as mouse muscle stem cells (satellite cells) and stable, immortalized cell lines derived therefrom. The recombinant peptide of the invention can be expressed in stable cell lines that contain the gene construct integrated into a chromosome or, in stable or transiently transfected cells, in the form of a plasmid.

Transgenic non-human animals as hosts transfected with, e.g. a gene gun, and/or expressing the nucleic acid molecule of the present invention also lie within the scope of the invention. In a preferred embodiment, the transgenic animal is a mammal, e.g. a hamster, mouse, rat, cow, cat, pig, dog, horse, rabbit or monkey. Transgenic plants as hosts transfected with and/or expressing the nucleic acid molecule of the present invention also lie within the scope of the present invention.

In yet another embodiment, the present invention relates to a method of producing a peptide of the invention comprising culturing the host cell of the invention under suitable conditions and isolating the peptide produced.

A large number of suitable methods exist in the art to produce peptides in appropriate hosts. If the host is a unicellular organism such as a prokaryote or a mammalian or insect cell, the person skilled in the art can revert to a variety of culture conditions. Conveniently, the produced protein is harvested from the culture medium, lysates of the cultured cells or from isolated (biological) membranes by established techniques. In the case of a multicellular organism as a host comprising multiple cells carrying the nucleic acid of the invention, a fraction of these cells may serve as source for the peptide of the invention, for example said fraction may be the harvestable part of a plant. A preferred method involves the synthesis of nucleic acid sequences c by PCR and its insertion into an expression vector. Subsequently a suitable host may be transfected or transformed with the expression vector. Thereafter, in the case that the host is a cell, the host is cultured to produce the desired peptide, which is isolated and purified.

Appropriate culture media and conditions for the above-described host cells are known in the art. For example, suitable conditions for culturing bacteria are growing them under aeration in Luria Bertani (LB) medium. To increase the yield and the solubility of the expression product, the medium can be buffered or supplemented with suitable additives known to enhance or facilitate both. E. coli can be cultured from 4 to about 37° C., the exact temperature or sequence of temperatures depends on the molecule to be overexpressed. In general, the skilled person is also aware that these conditions may have to be adapted to the needs of the host and the requirements of the peptide or protein expressed. In case an inducible promoter controls the nucleic acid of the invention in the vector present in the host cell, expression of the polypeptide can be induced by addition of an appropriate inducing agent. Suitable expression protocols and strategies are known to the skilled person.

Depending on the cell type and its specific requirements, mammalian cell culture can e.g. be carried out in RPMI or DMEM medium containing 10% (v/v) FCS, 2 mM L-glutamine and 100 U/ml penicillin/streptomycin. The cells can be kept at 37° C. in a 5% CO₂, water saturated atmosphere.

Suitable media for insect cell culture is e.g. TNM+10% FCS or SF900 medium. Insect cells are usually grown at 27° C. as adhesion or suspension culture.

Suitable expression protocols for eukaryotic cells are well known to the skilled person and can be retrieved e.g. from in Sambrook, 2001.

Such methods are well known in the art (see, e.g., Sambrook et al., supra).

An alternative method for producing the peptide of the invention is in vitro translation of mRNA. Suitable cell-free expression systems for use in accordance with the present invention include rabbit reticulocyte lysate, wheat germ extract, canine pancreatic microsomal membranes, E. coli S30 extract, and coupled transcription/translation systems such as the TNT-system (Promega). These systems allow the expression of recombinant peptides or proteins upon the addition of cloning vectors, DNA fragments, or RNA sequences containing coding regions and appropriate promoter elements.

Methods of isolation of the peptide produced are well-known in the art and comprise, without limitation, method steps such as ion exchange chromatography, gel filtration chromatography (size exclusion chromatography), affinity chromatography, high pressure liquid chromatography (HPLC), reversed phase HPLC, disc gel electrophoresis or immunoprecipitation (see, for example, Sambrook, 2001).

The present invention also relates to a peptide encoded by the nucleic acid molecule of the present invention or obtainable by the method of the present invention.

The peptide of the present invention can also be produced synthetically. Chemical synthesis of peptides is well known in the art. Solid phase synthesis is commonly used and various commercial synthesizers are available, for example automated synthesizers by Applied Biosystems Inc., Foster City, Calif.; Beckman; MultiSyntech, Bochum, Germany etc. Solution phase synthetic methods may also be used, although it is less convenient. Functional groups for conjugating the peptide of the invention to small molecules, label moieties, peptides, or proteins thus forming the fusion molecule of the invention may be introduced into the molecule during chemical synthesis. In addition, small molecules and label moieties/reporter units may be attached during the synthetic process. Preferably, introduction of the functional groups and conjugation to other molecules minimally affects the structure and function of the subject peptide.

Furthermore, the peptide of the invention may also be produced semi-synthetically, for example by a combination of recombinant and synthetic production. In the case that fragments of the peptide are produced synthetically, the remaining part of the peptide would have to be produced otherwise, e.g. recombinantly, and then linked to the fragment to form the peptide of the invention.

In a preferred embodiment, the peptide of the invention is attached to a linker. Regarding the structure and position of the linker, the same applies as already described above. The linker is preferably located at the N-terminus of the peptide. It is also preferred that the linker is a lysine.

In another preferred embodiment, a disulfide bond is present between the cysteines at positions 1 and 7 or 7 and 8 of SEQ ID NO: 2.

The absence or presence of disulfide bonds can be advantageous for peptides to maintain their biological activity and conformational stability. The present inventors have surprisingly found that not only cysteines present in the peptide of the invention are necessary for the peptide to exert its internalizing activity, but also that disulfide bonds between two out of the three cysteines further promote the internalizing activity of the peptide.

As shown in example 3, the presence of disulfide bonds between the cysteines at positions 1 and 7 as well as positions 7 and 8 further increase the internalizing properties of the peptide of the invention. Accordingly, not only the presence but also the position of said disulfide bond appears to be advantageous for the internalizing activity of the peptide of the invention.

As well known in the art, disulfide bonds can be selectively introduced into a peptide e.g. during solid phase synthesis by the application of appropriate protecting groups to cysteines which should not form disulfide bonds.

In a preferred embodiment, the peptide of the invention is modified. Exemplary modifications include esterification, glycosylation, acylation such as acetylation or linking myristic acid, amidation, phosphorylation, biotinylation, PEGylation, coupling of farnesyl and similar modifications which are well known in the art. Modifications also called derivatizations can be effected at the N-terminus, the C-terminus or at any amino acid in between (e.g. farnesyl coupling to a Cys side chain). It is preferred that the C-terminus of the peptide of the invention is modified, preferably by amidation. It is believed that such modifications enhance the stability of the peptide to exogenous peptidases. Conversion of the acid function on the C-terminus into an aldehyde is used for chemoselective ligation or the formation of reduced peptide bonds in peptidomimetics.

Methods for acylating, and specifically for acetylating the free amino group at the N-terminus are well known in the art. For the C-terminus, the carboxyl group may be modified, by esterification with alcohols or amidated to form-CONH₂ or CONHR. Methods of esterification and amidation are done using well known techniques.

The present invention furthermore relates to a fusion molecule comprising the peptide of the invention.

A fusion molecule, in the context of the present invention is an at least bipartite molecule comprising the peptide of the invention forming one moiety coupled to at least one other moiety, as has been defined above. The peptide and the at least one other moiety may be separated by a linker as described above forming an additional moiety or may be directly coupled. As shown in the examples (see Example 4), the peptide of the invention can exert its internalizing activity when fused to another moiety with or without using a linker separating both moieties. It is preferred that the fusion molecule essentially retains the internalizing activity of the peptide of the invention. Preferably at least 50% of the internalizing activity is retained, more preferably at least 60%, even more preferably at least 70%, at least 80% or at least 90%, most preferably at least 95% or 100% of the internalizing activity of the peptide of the invention is retained in the fusion molecule.

The at least one other moiety may be fused to the peptide of the invention at the N-terminus, the C-terminus or, if applicable, to any amino acid other than the terminal amino acids. It is preferred that the at least one other moiety is fused to the N-terminus, optionally separated by a linker as described above. Additional moieties may be fused to the moiety already comprised in the fusion molecule or to the C-terminus or to an amino acid other than the terminal amino acids of the peptide of the invention. The skilled person is well aware of tests how to define the optimal order and/or combination of moieties in the fusion molecule of the invention. Exemplary tests include the evaluation of the internalizing activity of a fusion molecule of interest according to the invention as described further below and optionally comparing said activity with that of the peptide of the invention or with other fusion molecules according to the invention having a different order of the fused moieties.

The fusion molecule according to the invention comprising the peptide of the invention fused to another peptide or polypeptide does not include fusion molecules, wherein the fusion results in naturally occurring peptides or polypeptides such as crotamine. In other words, a peptide or polypeptide fused to the peptide of the invention is of heterologous origin, i.e. is derived from a peptide or polypeptide different from crotamine.

In case of a fusion molecule comprising a peptide or polypeptide fused to the peptide of the present invention, it is preferred that said peptide or polypeptide is fused to the N-terminus of the peptide of the invention. In a more preferred embodiment, the N-terminus of the resulting fusion molecule is modified as described above, e.g. by acylation such as acetylation.

The fusion molecule of the present invention can be produced and isolated according to the methods described above for the production of the peptide of the invention.

In a preferred embodiment, the peptide of the invention is fused to a nucleic acid, a peptide or a polypeptide, an aptamer, a small molecule, a nanoparticle or nanocarrier or a contrast agent.

The nucleic acid can be linear or circular, e.g. in the form of a plasmid, an antisense RNA or siRNA. In this regard, the above described nucleic acid mimicking molecules, e.g. PNA, are also suitable as moieties.

Aptamers are DNA or RNA molecules that have been selected from random pools based on their ability to bind other molecules. Aptamers include those which bind nucleic acid, proteins, small organic compounds, and even entire organisms such as unicellular organisms. A database of aptamers is maintained at http://aptamer.icmb.utexas.edu/.

More specifically, aptamers can be classified as DNA or RNA aptamers or peptide aptamers. Whereas the former consist of (usually short) strands of oligonucleotides, the latter consist of a short variable peptide domain, attached at both ends to a protein scaffold.

Examples of peptides fused to the peptide of the invention include pro-apoptotic peptides such as the SmacN7 peptide having the sequence AVPIAQK (see the examples) which is derived from the N-terminus of Smac. Further examples comprise p53 or p53-derived peptides as well as p21 or p21-derived peptides which are involved in the regulation of apoptosis and might be useful in the treatment of cancer. ICAM-1 or IKKβ and/or üeütides derived therefrom are used in inflammation studies and treatment. The peptide of invention can also be fused to polypeptides, e.g. enzymes such as thymidine kinase, neuroamidase, HSP70 or GFP.

Nanoparticles (or nanopowders or nanoclusters or nanocrystals or nanocarriers) are microscopic particles with at least one dimension less than 100 nm. Nanocarriers, usually having a diameter of about 50 to 500 nm, are able to house smaller molecules, especially drugs which can then be delivered at the desired site. Nanocarriers existing to date are temperature and/or pH sensitive, which means that they can release their cargo upon heating or a change in the pH. They are mostly structurally stable in the normal physiological environment and resistant to e.g. intravenous administration. Polymeric core-shell nanocarriers are small in size (generally less than 200 nm), with shells that protect enclosed bioactive compounds against degradation and digestive fluids.

In a preferred embodiment of the fusion molecule of the present invention, the peptide of the invention is fused to a reporter unit. Suitable reporter units are well-known to the skilled person and comprise fluorescent dyes such as FITC or TAMRA or reporter units for MRI or PET such as Gd-DOTA-, Gd-DTPA-, ⁶⁴Cu-DOTA- or ⁶⁸Ga-DOTA, or nucleic acids such as siRNA, antisense oligonucleotides, PNA or nucleic acids encoding reporter genes, preferably operably linked to a regulatory element, such as a promoter, or contrast agents for other imaging techniques.

In another preferred embodiment of the fusion molecule of the present invention, the peptide of the invention is fused to a pharmacologically active compound. Pharmacologically active compounds may belong to different substance classes such as e.g. nucleic acids, peptides, small molecules, nanoparticles etc. as listed above. Exemplary compounds are anticancer drugs, antibiotics or compounds used in the treatment of other diseases as described further below.

In case the peptide of the invention is fused to more than one other moiety, suitable combinations of moieties can be e.g. a linker moiety and a pharmacologically active moiety, or a detectable moiety and a pharmacologically active moiety, optionally further comprising one or more linker moieties.

In a preferred embodiment, the peptide or fusion molecule is applied for research and/or diagnostic purposes.

The peptide or fusion molecule of the present invention can be applied e.g. in optical imaging methods, magnetic resonance imaging (MRI), primarily used in medical imaging to demonstrate pathological or other physiological alterations of living tissues, or positron emission tomography (PET), a nuclear medicine imaging technique which produces a three-dimensional image or map of functional processes in the body.

In MRI, a contrast agent may be applied to improve the measurement. The agent may be as simple as water, taken orally, e.g. for imaging the stomach and small bowel although substances with specific magnetic properties may also be used. Most commonly, a paramagnetic contrast agent (usually a gadolinium compound) is given. This provides high sensitivity for e.g. the detection of vascular tissues (e.g. tumors) and permits assessment of brain perfusion (e.g. in stroke).

More recently, superparamagnetic contrast agents (e.g. iron oxide nanoparticles) have become available. These may be used for liver imaging—normal liver tissue retains the agent, but abnormal areas (e.g. scars, tumors) do not. They can also be taken orally, to improve visualisation of the gastrointestinal tract, and to prevent water in the gastrointestinal tract from obscuring other organs (e.g. pancreas). Diamagnetic agents such as barium sulfate have been studied for potential use in the gastrointestinal tract, but are less frequently used.

To conduct a PET scan, a short-lived radioactive tracer isotope, which decays by emitting a positron and which also has been chemically incorporated into a metabolically active molecule, is injected into the living subject (usually into the blood circulation). During a waiting period, the metabolically active molecule becomes concentrated in the tissues of interest; then the research subject or patient is placed in the imaging scanner. The molecule most commonly used for this purpose is fluorodeoxyglucose (FDG), a sugar, for which the waiting period is typically an hour.

The peptide of the present invention may serve as shuttle in order to target the respective contrast agent coupled thereto to the site of interest in both methods. The intracellular delivery should provide a higher sensitivity as well as specificity as compared to known commercially available contrast agents. Aside of the more specific detection of targeted cells (e.g. in cancer diagnosis or the detection of loss of β-cells in diabetes) such contrast agents can be used for non-invasive tracking of the fate and action of transplanted cells in cellular therapies (e.g. cancer treatment with cytotoxic T-cells). Cell based therapies such as stem cell therapies or adoptive immunotherapies were already successfully tested and also monitored in animal models, mainly by invasive optical imaging methods (e.g. Sauer M G, 2004). However, the clinical application still suffers from the lack of a non-invasive diagnostic method for a long-term quantitative and qualitative evaluation of the transplanted cells. This is essential for the monitoring of the treatment and its efficacy. By a non-toxic labeling procedure (most preferable repeatedly applicable) with specifically targeted contrast agents cells can be followed in vivo, and their accumulation as well as function can be monitored using imaging techniques like MRI, nuclear or optical imaging.

The present invention furthermore relates to a pharmaceutical composition comprising the peptide of the invention or the fusion molecule of invention.

The term “composition”, as used in accordance with the present invention, relates to a composition which comprises at least one of the recited compounds. The composition may be in solid or liquid form and may be, inter alia, in the form of (a) powder(s), (a) tablet(s), (a) solution(s) or (an) aerosol(s).

In accordance with the present invention, the term “pharmaceutical composition” relates to a composition for administration to a patient, preferably a human patient. The pharmaceutical composition of the invention comprises the compounds recited above, alone or in combination. It may, optionally, comprise further molecules capable of altering the characteristics of the compounds of the invention thereby, for example, stabilizing, modulating and/or activating their function. The pharmaceutical composition of the present invention may, optionally and additionally, comprise a pharmaceutically acceptable carrier. By “pharmaceutically acceptable carrier” is meant a non-toxic solid, semisolid or liquid filler, diluent, encapsulating material or auxiliary formulation of any type. Examples of suitable pharmaceutical carriers are well known in the art and include phosphate buffered saline solutions, water, emulsions, such as oil/water emulsions, various types of wetting agents, sterile solutions, organic solvents including DMSO etc. Compositions comprising such carriers can be formulated by well known methods. Generally, the formulations are prepared by contacting the components of the pharmaceutical composition uniformly and intimately with liquid carriers or finely divided solid carriers or both. Then, if necessary, the product is shaped into the desired formulation.

In a preferred embodiment, the composition is suitable for parenteral administration. The term “parenteral” as used herein refers to modes of administration, which include intravenous, intramuscular, intraperitoneal, intrasternal, subcutaneous and intraarticular injection and infusion. These pharmaceutical compositions will be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. Generally, the regimen as a regular administration of the pharmaceutical composition should be in the range of 1 μg to 5 g units per day. However, a more preferred dosage might be in the range of 0.01 mg to 100 mg, even more preferably 0.01 mg to 50 mg and most preferably 0.01 mg to 10 mg per day.

The components of the pharmaceutical composition to be used for therapeutic administration must be sterile. Sterility is readily accomplished by filtration through sterile filtration membranes (e.g., 0.2 micron membranes).

The components of the pharmaceutical composition ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous solution, and the resulting mixture is lyophilized. The infusion solution is prepared by reconstituting the lyophilized compound(s) using bacteriostatic Water-for-Injection.

Preservatives and other additives may also be present such as, for example, antimicrobials, anti oxidants, chelating agents, and inert gases and the like. Furthermore, the pharmaceutical composition may comprise further agents depending on its intended use.

The pharmaceutical composition may be particularly useful for the treatment and/or prevention of diseases, including for example cancer, enzyme deficiency diseases, infarcts or cerebral ischemia, diabetes, inflammatory diseases, infections such as bacterial, viral or fungal infections, autoimmune diseases such as systemic lupus erythematodes (SLE), multiple sclerosis or rheumatoid arthritis, diseases with amyloid-like fibrils such as Alzheimer's disease (AD) and Parkinson's disease (PD) or certain forms of myopathy. Further preferred diseases are those where the pharmaceutical composition has to cross restrictive membrane structures such as the blood-brain barrier in order to be effective. Examples of such diseases are AD or PD.

Cancer, in accordance with the present invention refers to a class of diseases or disorders characterized by uncontrolled division of cells and the ability of these to spread, either by direct growth into adjacent tissue through invasion, or by implantation into distant sites by metastasis (where cancer cells are transported through the bloodstream or lymphatic system).

Enzyme deficiencies leading to disorders are caused by one or more mutations in one or more enzymes in a cell including fatty acid oxidation disorders, urea cycle disorders, phenylketonuria, glycogen branching enzyme deficiency and mitochondrial enzyme deficiencies.

Bacterial infections, in accordance with the present invention include but are not limited to bacterial meningitis, cholera, diphtheria, listeriosis, pertussis (whooping cough), pneumococcal pneumonia, salmonellosis, tetanus, typhus or urinary tract infections.

Viral infections, in accordance with the present invention include but are not limited to mononucleosis, AIDS, chickenpox, common cold, cytomegalovirus infection, dengue fever, Ebola hemorrhagic fever, hand-foot and mouth disease, hepatitis, influenza, mumps, poliomyelitis, rabies, smallpox, viral encephalitis, viral gastroenteritis, viral encephalitis, viral meningitis, viral pneumonia or yellow fever.

Fungal infections in accordance with the present invention include but are not limited to aspergillosis, blastomycosis, candidiasis, coccidioidomycosis, cryptococcosis, histoplasmosis or tinea pedis.

Autoimmune diseases, in accordance with the present invention refer to diseases which arise from an overactive immune response of the body against substances and tissues normally present in the body. Autoimmune diseases are well known to the person skilled in the art and include, but are not limited to Lupus erythematosus, acute disseminated encephalomyelitis, aplastic anemia, autoimmune hepatitis, diabetes mellitus, multiple sclerosis, optic neuritis or rheumatoid arthritis.

Diseases with amyloid-like fibrils in accordance with the present invention are diseases which share as a common feature that the normally soluble peptide amyloid-beta or the protein alpha-synuclein aggregates into an ordered fibrillar structure typically resulting in increased oxidative injury, excitotoxicity and altered cell cycle. Diseases with amyloid-like fibrils include but are not limited to Alzheimer's disease (AD) and Parkinson's disease (PD).

Alzheimer's disease is a neurodegenerative disease characterized by progressive cognitive deterioration together with declining activities of daily living and neuropsychiatric symptoms or behavioral changes. It is the most common type of dementia.

Parkinson's disease is a degenerative disorder of the central nervous system that often impairs the sufferer's motor skills and speech.

Myopathies are neuromuscular diseases in which the muscle fibers do not function, resulting in muscular weakness. Several classes of myopathy are known and include but are not limited to for example muscular dystrophies, congenital myopathies, mitochondrial myopathies or inflammatory myopathies.

In a preferred embodiment of the present invention, the pharmaceutical composition is used as a vaccine to prevent, inter alia, bacterial, viral or fungal infections as described above. In a particularly preferred embodiment, the vaccine is a DNA vaccine. Accordingly, the peptide of the invention is coupled to a nucleic acid which, when introduced into a cell, will be translated and the resulting peptide or protein will be processed in order to be presented in the form of antigenic fragments on the surface of cells, wherein the latter are usually found in bacteria, fungi or viruses. Alternatively, the composition of the present invention comprising the peptide of the invention coupled to another moiety, such as a peptide or protein, also directly provides the cell with the expressed protein/peptide or the molecule against which immunity is to be induced when introduced into a cell without the need for it to be translated.

In the treatment of the above disorders and diseases, at least one pharmacologically active moiety is to be coupled to the peptide of the invention. As described above for moieties in general, pharmacologically active moieties may belong to different substance classes.

In addition to a pharmacologically active moiety, the fusion molecule of the invention may further comprise a targeting sequence or moiety specifically targeting the desired tissue or structure, as described further below for fusions molecules comprised in the diagnostic composition of the invention.

In addition, the present invention relates to a diagnostic composition comprising at least one of (a) the nucleic acid molecule of the invention, (b) the vector of the invention, (c) the peptide of the invention, or (d) the fusion molecule of the invention.

In accordance with the present invention, the term “diagnostic composition” relates to compositions for diagnosing individual patients for their potential response to or curability by the pharmaceutical compositions of the invention. Furthermore, a diagnostic composition can denote a substance comprising the peptide of the invention fused to at least one more moiety for research purposes as has already been described above for the peptide and the fusion molecule of the invention. The diagnostic composition may further comprise appropriate buffer(s). The diagnostic composition may be packaged in a container or a plurality of containers.

Preferred embodiments of the diagnostic composition comprise the peptide of the present invention fused to a reporter unit and a target sequence specifically targeting to the desired tissue or structure. For example, the target sequence can be an antisense oligonucleotide or a peptide sequence which is enzymatically cleavable. Such target sequences can be designed in order to deliver the reporter units to e.g. cancerous cells or tissues or otherwise diseased cells or tissues.

Using the diagnostic composition of the present invention, diseases such as cancer can be detected in an early stage thus enabling for an early and more promising therapy. In particular, the pH value in cancerous tissue is usually more acidic than in non-cancerous tissue. Accordingly, a reporter unit sensitive to changes in the pH value and thus capable of detecting tissue with an e.g. more acidic pH value, in connection with the diagnostic agent of the present invention may serve in the detection of cancer.

Another example refers to the detection of insulin mRNA in insulin producing β-cells by coupling the peptide of the present invention to an antisense nucleic acid binding to insulin mRNA. With this molecule as a diagnostic tool, the insulin production in β-cells can be monitored and the decrease of insulin production and/or loss of β-cells which may indicate an early stage of diabetes can be detected.

Yet a different example refers to the detection of transplanted, healthy cells in the body after a transplantation event to monitor the tolerance of the body towards the graft as well as the fate of transplanted cells in the body.

The above-described combinations of moities usable in the detection and/or diagnosis with therapeutically applicable moieties become more and more important in the context of theranostics. This is the term used to describe the proposed process of diagnostic therapy for individual patients, to test them for possible reaction to taking a new medication and to tailor a treatment for them based on the test results.

The present invention furthermore relates to a method of detecting the internalization behaviour of a fusion molecule comprising the peptide of the invention or a fusion molecule according to the invention, comprising (a) administering said fusion molecule to a cell and (b) detecting the internalization of the fusion molecule.

The present method is particularly suitable to estimate the applicability of the peptide of the present invention in connection with other moieties coupled thereto for medical or research purposes, e.g. in the form of pharmaceutical or diagnostic compositions. If efficient internalization of the peptide or the fusion molecule comprising more than one moieties is detected and, optionally, its localization is in the cytoplasm, this indicates that the respective molecule can be efficiently used for medical and research purposes.

In a preferred embodiment, the method further comprises (c) comparing said internalization with that of the peptide of the invention or with that of fusion molecules according to the invention having a different order of the fused moieties. A higher internalization as compared to other fusion molecules in this case indicates that the respective fusion molecule can be efficiently used for medical and research purposes.

In addition, the present invention relates to a method of treating, preventing or diagnosing a condition selected from cancer, enzyme deficiency diseases, infarcts, cerebral ischemia, diabetes, inflammatory diseases, infections such as bacterial, viral or fungal infections, autoimmune diseases such as systemic lupus erythematodes (SLE) or rheumatoid arthritis, diseases with amyloid-like fibrils such as Alzheimer's disease (AD) and Parkinson's disease (PD) or certain forms of myopathy comprising administering the composition of the invention to a subject in need thereof.

The present invention also relates to the peptide, the fusion molecule or the composition of the invention for therapeutic or diagnostic purposes. In a preferred embodiment the therapeutic purpose is the treatment and/or prevention of cancer, enzyme deficiency diseases, infarcts, cerebral ischemia, diabetes, inflammatory diseases, infections such as bacterial, viral or fungal infections, autoimmune diseases such as systemic lupus erythematodes (SLE) or rheumatoid arthritis, diseases with amyloid-like fibrils such as Alzheimer's disease (AD) and Parkinson's disease (PD) or certain forms of myopathy.

The present invention furthermore relates to a kit comprising at least one of (a) the nucleic acid molecule of the invention, (b) the vector comprising the nucleic acid molecule of the invention, (c) the host cell comprising the vector of the invention, (d) the peptide of the invention or (e) the fusion molecule or protein of the invention.

The various components of the kit may be packaged in one or more containers such as one or more vials. The vials may, in addition to the components, comprise preservatives or buffers for storage.

The figures show:

FIG. 1: Comparison of the intracellular fluorescence of the synthesized peptide fragments (cargo: K(FITC), peptide 23=SEQ ID NO: 2). corr. f.u.=corrected fluorescence units (measured cell-related FITC fluorescence corrected for the number of cells evaluated by Bisbenzimid 33342 fluorescence.

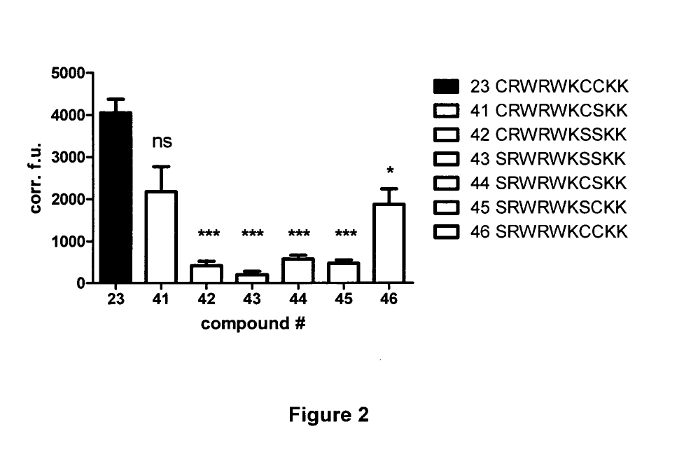

FIG. 2: Influence of substitution of cysteines by serine in peptide 23 (SEQ ID NO: 2) on the internalization efficiency. ns, not significant; *, p<0.05, ***, p<0.001 significantly different compared to peptide 23 (ANOVA, Bonferroni's Multiple Comparison Test). K(FITC) was coupled to all peptides at the N-terminus.

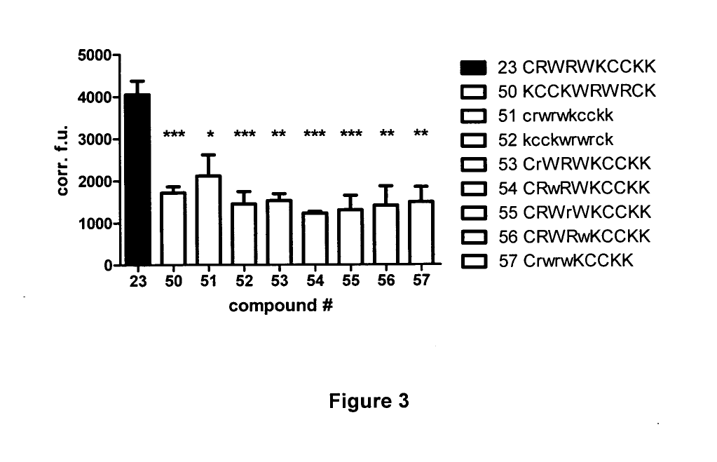

FIG. 3: Influence of sequence order and amino acid chirality on the internalization efficiency. *, p<0.05, **, p<0.01, ***, p<0.001 significantly different compared to peptide 23 (ANOVA, Bonferroni's Multiple Comparison Test). K (FITC) or k (FITC) was coupled to all peptides at the N-terminus.

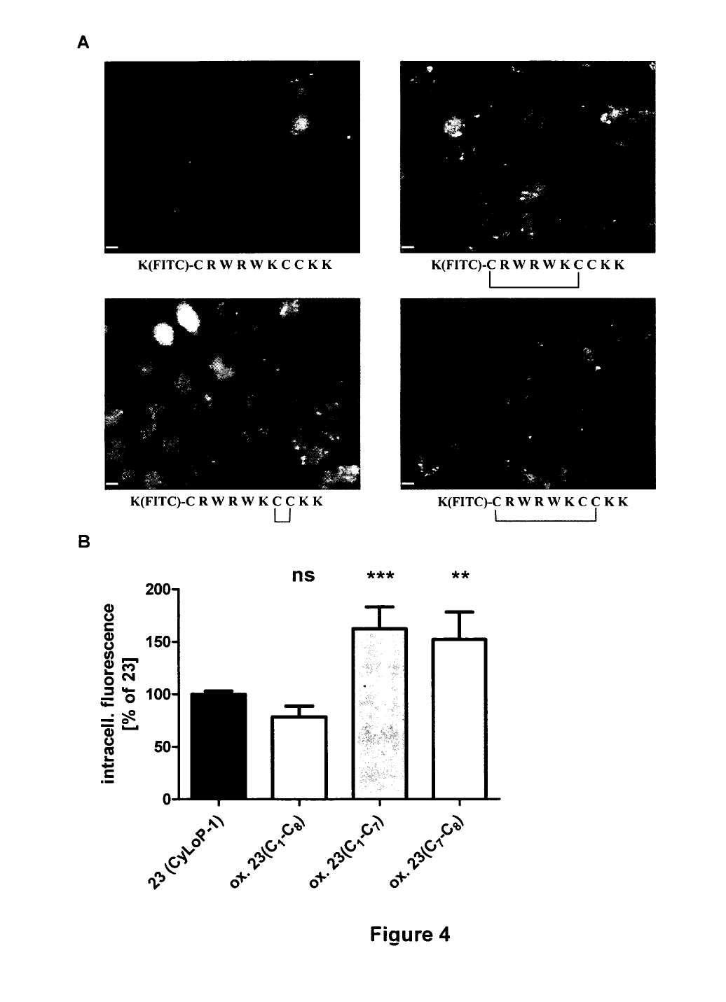

FIG. 4: Influence of intracellular disulphide bridges after controlled oxidation of peptide 23 (CyLoP-1, SEQ ID NO: 2) on the internalization efficiency. (A) Fluorescence microscopic images. (B) Fluorescence spectroscopic quantification. ns, not significant; **, p<0.01, ***, p<0.001 significantly different compared to peptide 23 (ANOVA, Bonferroni's Multiple Comparison Test). K(FITC) was coupled to all peptides at the N-terminus.

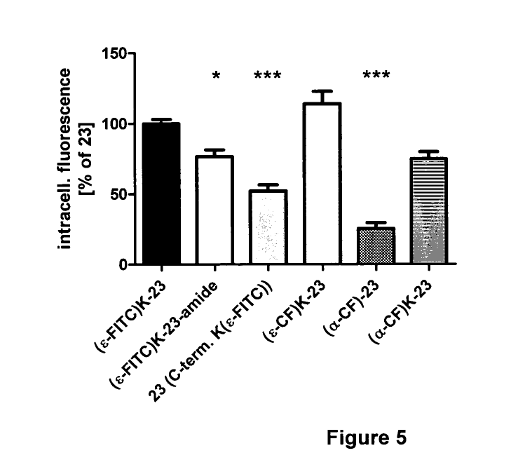

FIG. 5: Influence of the functionality at the C-terminus and position and type of fluorophore coupling to peptide 23 (CyLoP-1, SEQ ID NO: 2) on the internalization efficiency. *, p<0.05, ***, p<0.001 significantly different compared to peptide 23 (ANOVA, Bonferroni's Multiple Comparison Test). Carboxyfluorescein (CF) or FITC were coupled as indicated. Peptide 23-amide contains an amide group at the C-terminus instead of a free acid group.

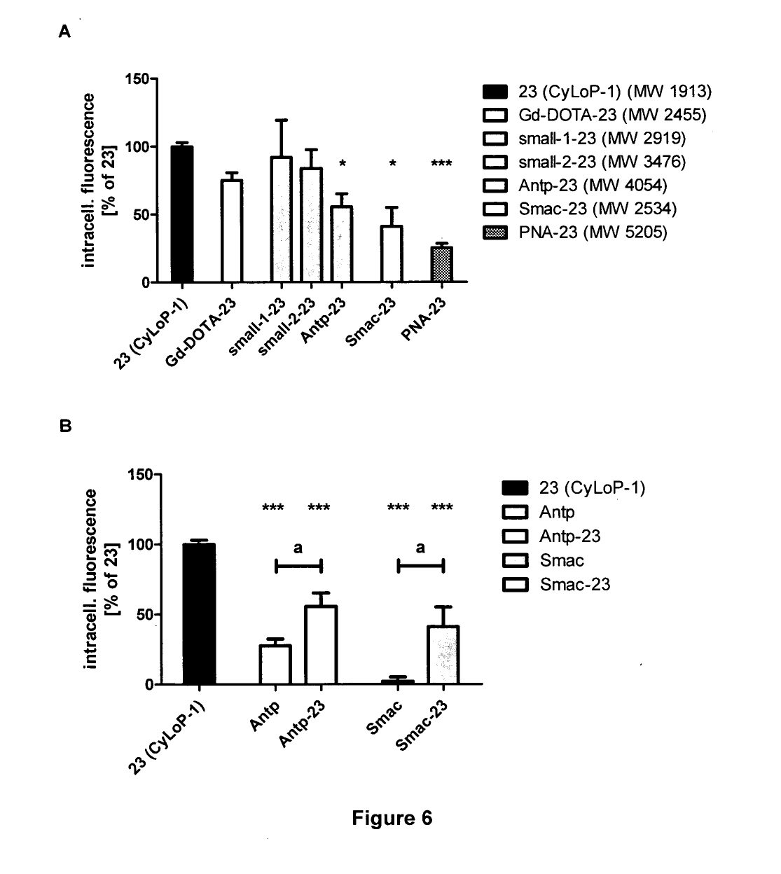

FIG. 6: Influence of cargo molecules coupled to the N-terminus of peptide 23 (CyLoP-1, SEQ ID NO: 2) via the lysine linker on the internalization efficiency. (A) Effect of various cargo molecules. (B) Comparison between peptide 23-cargo conjugates/fusions and the corresponding cargo alone.

*, p<0.05, ***, p<0.001 significantly different compared to peptide 23; a, p<0.05 significantly different pairs (ANOVA, Bonferroni's Multiple Comparison Test).

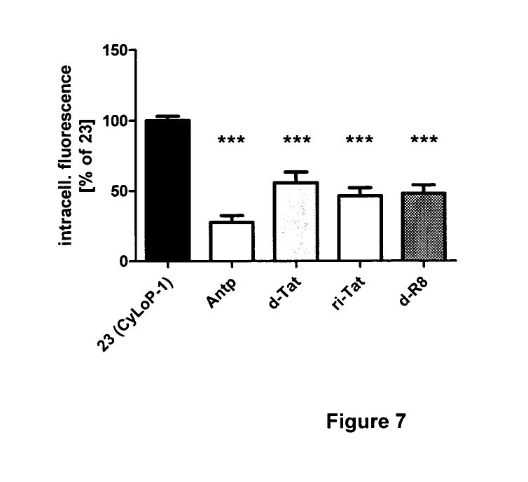

FIG. 7: Comparison of the internalization efficiency of peptide 23 (CyLoP-1, SEQ ID NO: 2) with four other known CPP. Antp, Penetratin (RQIKIWFQNRRMKWKK); d-Tat, d-Tat₄₉₋₅₇ (rkkrrqrrr); ri-Tat, d-Tat₅₇₋₄₉ (rrrqrrkkr); d-R8, octaarginine (rrrrrrrr); ***, p<0.001 significantly different compared to peptide 23 (ANOVA, Bonferroni's Multiple Comparison Test). K(FITC) or k(FITC) was coupled to all peptides at the N-terminus.

The invention will now be described by reference to the following examples which are merely illustrative and are not to be construed as a limitation of scope of the present invention.

### EXAMPLE 1

### Peptide Synthesis: Materials and Method

All solvents were of peptide synthesis grade. N,N-dimethylformamide (DMF), dichloromethane (DCM), trifluoroacetic acid (TFA), fluorescein isothiocyanate (FITC) and methanol were purchased from Acros Organics, Belgium. Protected Fmoc amino acid as well as resin was obtained from Novabiochem (Nottingham, UK). The side chain of lysine was protected by Boc or Dde, Cys by Trt or tBu, Arg by Pbf and, Trp by Boc.

### (A) Automatic Peptide Synthesis:

Peptides were prepared by fully automated solid-phase peptide synthesis using Fmoc/tBu-strategy and α-Fmoc-(ε-BOC)-lysine-TCP-polystyrene resin. The resin was distributed in 30 mg aliquots (15 μmol) to filter tubes, which were positioned in the format of a microtiter plate on valve blocks. Fmoc deprotection were carried out two times, 10 min. each, with 30% piperidine in dimethylformamide (DMF) (300 μl). Nine washing steps were done with DMF (300 μl). Fmoc-amino acids (0.5 M) were dissolved with 1-hydroxybenzotriazole (HOBt) (0.5 M) in DMF. Diisapropylethylamine [3 M in NMP, 60 μl] was added to the reaction vessels. Coupling reagents diisopropylcarbodiimide [3 M in DMF, 50 μl first coupling] or 1-H-benzotriazolium-1-[bis(dimethylamino)methylene]-5-chloro-tetrafluoroborate (1-), 3-oxide [TCTU, 0.5 M in DMF, 200 μl second coupling] and Fmoc-amino acids [first coupling 200 μl; second coupling 100 μl] were distributed to the reaction vessels. After 1 h coupling time, coupling reagents were filtered off and the resins were washed with DMF [1×200 μl] followed by a second coupling step (1 h). Amino acids were introduced using a sevenfold molar excess of the respective Fmoc-L-amino acid. After washing steps [4×400 μl] Fmoc deprotection was carried out two times, 10 min. each, with 30% piperidine in DMF (300 μl). Nine washing steps were done with DMF (300 μl). The coupling process was repeated according to the length of the peptide. The ε-group of N-terminal lysine was labeled manually with FITC (4 fold excess) with triethylamine (1:2) in DMF overnight. Peptides were washed with DMF, DCM, methanol (4× each) and dried before cleaving from the resin.

The peptides were cleaved off the resin and side-chain deprotected with Reagent K (500 μl).

### (B) Manual Peptide Synthesis:

The synthesis of selected peptides was carried out by solid phase Fmoc chemistry using a manual multiple peptide synthesizer (Heidolph Synthesis 1 synthesizer). Peptides were synthesized on α-Fmoc-(ε-BOC)-lysine-2-chlorotrityl resin (0.83 mmol/g). Fmoc protected amino acids (4 fold excess) were coupled with DIC/HOBT activation for 60 min. Fmoc was removed by 30% piperidine in DMF (2×10 min). Resin was washed after each cycle of coupling and deprotection with DMF (4×). Completeness of coupling and deprotection was monitored by Kaiser Test (38). Peptides were washed with DMF, DCM, methanol (4× each) and dried. The peptides were cleaved off the resin and side-chain deprotected with Reagent K (500 μl).

This cleavage reaction was optimized further on, in particular for the synthesis of DOTA based compounds, by using TFA: Reagent K: TMSBr (8.5:1.45:0.05) for 2 hr instead of Reagent K alone. For the oxidized peptides a different cleavage cocktail consisting of TFA:TIPS:H₂O was used for 1 h followed by addition of DMSO (5:1) at 0° C. for 30 min and stirring for 1 h at RT.

### EXAMPLE 2

### Purification and Characterization

All peptides were precipitated with MTBE. Precipitates were collected by centrifugation and resuspended in cold MTBE 2 times and were lyophilized.

Samples were purified by semipreparative RP-HPLC at RT using water/0.1% TFA (solvent A) and Acetonitrile/0.1% TFA (solvent B) on a Varian Polaris C18-Ether column (21.2 mm in diameter, length of 250 mm, particle size 5 μm). The product containing fractions were defined by investigation with analytical RP-HPLC on a Varian Polaris C18-Ether column (4.6 mm in diameter, length of 250 mm, particle size 5 μm). Mass spectrometry (ESI-MS) was used for further characterization. The purified product was dissolved in water and tert.butyl alcohol (1:4) with 2% acetic acid, and lyophilised.

Peptides containing cysteine are prone to oxidative formation of disulfide bonds, which could be formed intramolecularly, resulting in a cyclic peptide, or intermolecularly, forming oligomers or aggregates.

As this CPP is a cysteine rich peptide, care has to be taken during synthesis and storage of the peptides. During the synthesis of peptide, choice of protecting groups and proper scavengers during cleavage should be optimized. After lyophilization peptides were stored under nitrogen. Resolved samples for internalization studies were aliquoted and stored at −80° C. Solutions for cell studies were freshly prepared for each experiment from these aliquots.

### EXAMPLE 3

### SAR Studies

Various fragments derived from the proposed fragment (Cro₂₇₋₃₉) were synthesized to achieve an optimal fragment maintaining the distinctive features of the parent sequence. The original fragment Cro₂₇₋₃₉ (peptide 1, see table 1) is a peptide of 13 amino acid including synthetically difficult amino acids like methionine or cysteine. The aim of this study was to simplify the sequence maintaining the distinctive features of the parent sequence Cro₂₇₋₃₉. To visualize this aim series of peptides were synthesized by substituting or deleting amino acid residues but preserving the basic amino acids in the sequence. K (FITC) or k (FITC) was coupled to all peptides at the N-terminus to measure the internalization efficiency.

### (A) Substitution of Cysteine:

The purpose of substitution was to understand the importance of cysteine in the sequence. Replacement of cysteines one by one or all together by aminobutyric acid or serine will help in estimating the number and position of cysteines responsible for maintaining penetration ability and to minimize the complexity of the synthesis by avoiding cysteines.

### (B) Deletion of Cysteines:

The same rationale was valid studying the possibilities of avoiding cysteines completely. In the way of doing so we tried to delete cysteines from the sequence one by one and studying the effect of the deletion on the internalization efficiency.

### (C) Shortening the Sequence:

To find the minimal amino acid composition necessary for the best uptake, combinations were designed by deleting amino acids from the N-terminus keeping the positively charged amino acids the same. On the other hand, deletion of the positively charged amino acids from C-terminus was also studied.

### (D) Shortening the Sequence and Substitution:

Other variations include combined substitution and deletion to study the combined effect of the two. This included substitution of the cysteines by amino butyric acid and deletion from N-terminal amino acids one by one (until reaching the first cysteine). The idea was to check the appropriate length and the number of cysteine residues needed.

### (E) Substitution of Tryptophan:

Tryptophans are known to be prone to side reactions like alkylation. Also blends of tryptophans and cysteines are not the most favorable ones as there are more chances of side reactions during the course of disulfide formation by various methods chemically. Therefore tryptophan was substituted by proline or phenylalanine one by one or completely.

### (F) Effect of Chirality:

Screening of a number of fragments for cellular uptake led to a peptide shorter in length (peptide 23, SEQ ID NO: 2). Studies were carried on this peptide in order to check factors other than the amino acid composition itself.

Other distinctive features are the influence of the chirality of the peptide backbone as well as the sequence order on the cellular uptake. Peptides with d-isomers (whole sequence with D-amino acids), L-amino acids in reversed order and with D-amino acids in reversed order were synthesized. In addition, the effect of stereochemistry was studied only in the region of Trp-Arg-Trp-Arg by using single D-amino acids or exchanging all four residues. This pattern seemed to be important as indicated by the results of the initial screening.

### (G) Introduction of a Spacer Unit:

Studies show the importance of cysteines in the proposed fragment and their oxidation status. To extend our study in this direction attempts were made focusing on oxidation of the proposed fragment. In SEQ ID NO: 2 two cysteines are adjacent to each other. Investigations were carried out to estimate the favorable conditions for disulfide bond formation between them. The purpose of introduction of the spacer glycine was not only to study the necessity of an optimal distance between two cysteines for disulfide linkage but also to facilitate the synthesis of defined disulfide bonds through fragment condensation. Additionally, the effect of elongating the sequence by introducing an additional cysteine was studied for the convenient synthesis of peptides with two defined disulfide linkages.

Results of the experiments are depicted in FIGS. 1 to 3.

### (H) Controlled Oxidation of Cysteines (Intracellular Disulfide Bridges):

Cysteine residues in peptides can modify the biological activity of the peptide by their ability to form intra- and intermolecular bridges and hence promote oligomerization (Andreu, 2004). Thus, different oxidized forms of the peptide of the invention represented by SEQ ID NO: 2 (in the following also called CyLoP-1) and fused to lysine(FITC) were synthesized with defined intracellular cystine bridges.

In case of peptide 23 (CyLoP-1), three peptides with an intracellular disulfide bond between C₁-C₇, C₁-C₈, or C₇-C₈ are possible. Peptides were synthesized as mentioned in Example 1(B). Defined disulfide bridges were attained by the procedure described elsewhere (Wacker, 2008). The peptides were cleaved from the resin by 95% trifluoroacetic acid (TFA), 2.5% triisopropylsilane, 2.5% water. Analysis and purification conditions same as before (Example 2).

Two out of three oxidized peptides (disulfide bridges between C₁-C₇ and C₇-C₈) showed improved uptake compared to CyLoP-1 (FIG. 4) indicating that the redox status of the cysteines (or their potential oxidation in biological fluids) during the incubation of cells might play a crucial role in uptake. This is further supported by the fact that addition of an access of Dithiothreitol (DTT) to the incubation medium containing CyLoP-1 reduced the uptake as well as the cytosolic diffused fluorescence.

### EXAMPLE 4

### Effect of Cargos on Uptake

CPPs are known to be the carrier of various cargos through the plasma membrane. Cellular uptake of various cargos was studied. Cargos were linked to the peptide through a linker which can be varied according to the need. In this study lysine is used as a linker. Choice of the amino protecting groups depends on the requirement of the synthesis.

### Fluorescent Dye as a Cargo:

Coupling of FITC to ε-Amino Group:

For structure activity studies all peptides were coupled to FITC through a Boc-Lysine (Fmoc)-OH as a linker. The ε-group of N-terminal lysine was labeled with FITC (4 fold excess) with triethylamine (1:2) in DMF overnight.

Analysis and purification conditions are same as before (C).

Coupling of TAMRA and Carboxyfluorescein:

Different dyes were coupled at ε-amino group to study the effect of cargo on internalization. Different colored dyes were synthesized for the chasing experiments to study the mechanism of internalization. TAMRA was coupled both at α-amino and ε-amino group to study the effect of the position of the cargo on internalization. Coupling of these dyes were done by DIC, HOBt activation for 8 h.

Analysis and purification conditions are same as before (C).

### Position of the Fluorophore:

In order to ensure an optimal attachment of the fluorophore different positions and types of coupling of the dye were tested. A first criterion was the comparison of C-terminal and N-terminal labeling. FITC was attached to the side chain of the lysine by the method described before. A decrease in the intracellular uptake and cytosolic distribution was observed in case of C-terminal labeling.

Further comparison of labeling of the alpha amino group and the side chain amino group of lysine from N-terminus was carried out by coupling with carboxyfluorescein (CF). In this case FITC was exempted as coupling of FITC at the N-terminus will cause Edman degradation thus losing the defined compound.

In addition, carboxyfluorescein was coupled directly to the alpha amino group of the N-terminal cysteine in CyLoP-1 without using lysine as a linker.

Coupling of fluorophore was done as described in Example 1 (B). Analysis and purification conditions same as before (Example 2).

### Functional Group at the C-Terminus:

The choice of the C-terminal functional group also plays an important role in determining the stability of the peptide to exogenous peptidases. In this concern CyLoP-1 was synthesized with C-terminal acid and amide.

### Dual Labeling:

In order to determine the mechanism of internalization and to explore the reason for cytoplasmic gain CyLoP-1 was synthesized with dual fluorophores FITC and TAMRA at the C- and N-terminus respectively.

Coupling of the fluorophore was done as described before. Analysis and purification conditions same as before (Example 2).

Whereas coupling of CF or FITC via a lysine linker to the N-terminus had no significant influence on the uptake (FIG. 5) C-terminal FITC labeling as well as the direct coupling of the fluorophore to the N-terminal cysteine markedly decreased the cellular uptake. The exchange of the acid functional group at the C-terminus by an amide group was also negatively influencing the internalization and distribution in the cells.

### MR Reporter as Cargo:

Gd(DOTA) as Cargo:

To extend the variations in the range of the cargos an attempt was made for the synthesis of bimodal intracellular contrast agents using proposed peptide as a carrier of Gd (DOTA) [as MR reporter] and FITC [fluorescent marker].

Synthesis of the peptide was done as mentioned before. Coupling of the cargo was carried out by coupling of Fmoc-Lys (Dde)-OH as the linker. DOTA was coupled by DIC, HOBt activation for 12 h at α-amino position (after Fmoc deprotection). Later FITC was coupled at ε-amino after Dde deprotection by 2% hydrazine in DMF 3 min×2. Analysis and purification conditions are same as before (C).

The actual concentration of the peptide was estimated by fluorescence spectroscopy to be ˜25%. Accordingly GdCl₃.6H₂O was added (1:1) at pH 6 50° C. for 18 h. Repurification was done to get rid of the excess gadolinium.

Analysis and purification conditions are same as before (C). Samples were dialyzed for 48 h to remove excess Gadolinium and other inorganic salts.

### PNA as a Cargo:

Peptide Nucleic Acids (PNA) are effectively being applied in the field of antisense imaging with the limitation of being impermeable to the plasma membrane. To overcome this hurdle they are generally coupled to the delivery, vectors like cell permeable peptides (e.g. polyarginines, Tat, penetratin etc.). Thus intracellular delivery of PNA conjugated/fused to CyLoP-1 was studied. The selected antisense PNA targets mRNA of the red fluorescent dsRed protein (Su et al, 2007). The PNA-peptide fusion was obtained by continuous Fmoc synthesis. The peptide was synthesized on a rink amide MBHA resin at 0.2 mmol/g scale by the method described in Example 1 (B). Fmoc-Lys(Dde)-OH was introduced as a linker for the further coupling of the fluorophore. The PNA chain was elongated by continuous coupling of the respective PNA monomers, HATU, DIPEA (1:0.9:2) 1 h, followed by acetylation at each step. Regular washing with DMF/NMP, DCM, methanol, DCM, DMF/NMP was done to ensure the removal of the reacting reagents from the reaction vessel. FITC was attached to the linker lysine after the removal of Dde by 2% hydrazine in DMF. Once the synthesis of the PNA-peptide fusion is complete, the resin is thoroughly washed by DMF, DCM, and methanol dried and cleaved by reagent K for 3 h.

The filtrate was collected and precipitated by MTBE 2 times. The pellet collected by centrifugation was lyophilized in water:tert-butanol (1:4) and 5% acetic acid. Analysis and purification conditions are the same as before (Example 2).

PNA-CyLoP-1 fusion:
Ac-tccgtgaacggc-K(FITC)-CRWRWKCCKK-CONH₂

### Peptide as Cargo:

Penetratin, a well known CPP derived from the third helix of antennapedia is known for the delivery of various types of molecules through the plasma membrane. Also penetratin shows a close homology with CyLoP-1 in context of the amino acid composition. On comparison of the delivery efficiency of penetratin and CyLoP-1, it was observed that CyLoP-1 was better taken up by cells retaining the cytosolic distribution at a concentration as low as 2.5 μM which was not the case for penetratin. In order to study the cumulative effect of the two peptides on the cell penetration a penetratin-CyLoP-1 fusion was synthesized. To evaluate the influence of the cargo/molecular weight on internalization efficacy two truncated penetratin sequences (small-1, small-2) were coupled to CyLoP-1 as well.

Fusions were synthesized by continuous solid phase synthesis by the method described in Example 1 (B). Each coupling was followed by capping to block the unreacted active sites. Fmoc-Lys(Dde)-OH was introduced as a linker in between CyLoP-1 and penetratin for the further coupling of the fluorophore. Upon completion of the synthesis the conjugate/fusion molecule was cleaved from the resin by reagent K for 3 h followed by precipitation in MTBE and centrifugation.

Analysis and purification conditions are the same as described before (Example 2).

Penetratin-CyLoP-1 fusion:
Ac-RQIKIWFQNRRMKWK-K(FITC)-CRWRWKCCKK
Small-1-CyLoP-1 fusion:
RMKWK-K(FITC)-CRWRWKCCKK
Small-2-CyLoP-1 fusion:
WFQNRRMKWK-K(FITC)-CRWRWKCCKK

### Smac as Cargo:

In another example CyLoP-1 was fused to a pro-apoptotic peptide derived from the N-terminus of the Smac protein. Smac (second mitochondria-derived activator of caspase) is released from mitochondria in response to apoptotic stimuli and promote caspase activation by binding to the inhibitor of apoptosis proteins (IAPs) and abolishes their inhibitory activity (Heckl, 2008). SmacN7 (AVPIAQK) the IAP binding sequence alone is impermeable to the plasma membrane therefore fusion with membrane permeable peptides is an easy way to accomplish targeting.

SmacN7 was coupled to CyLoP-1 by continuous SPPS by the methods discussed in Example 1 (B). The fluorophore was also attached to these fusion peptides to trace the intracellular localization.

Analysis and purification conditions are the same as described before.

Smac CyLoP-1 fusion: Ac-AVPIAQK-(FITC)-CRWRWKCCKK

After coupling of larger cargo molecules via the lysine linker to the N-terminus of CyLoP-1, a decrease of internalization was observed depending on size as well as the type of cargo (FIG. 6A). The fusion molecule with a peptide nucleic acid showed the lowest intracellular fluorescence. Nevertheless, conjugation of CyLoP-1 was clearly enhancing the uptake into cells compared to cargo molecules alone (FIG. 6B) proving its ability to transport bioactive molecules into the cytosol.

### EXAMPLE 5

### Cell Culture

NIH 3T3 mouse fibroblast cells were cultured as a monolayer at 37° C. with 10% CO₂ in Dulbecco's Modified Eagle's Medium (DMEM) supplemented with 10% fetal bovine serum, 4 mM L-glutamine, 100 μg/mL streptomycin and 100 U/mL penicillin (all purchased from Biochrom AG, Germany). Cells were passaged by trypsinization with trypsin/EDTA 0.05/0.02% (w/v) in phosphate-buffered saline (PBS; Biochrom AG, Germany) every second to third day.

### Evaluation of Internalization in Additional Cell Lines

### Cell Lines Examined:

C6 rat glioma
N18 mouse neuroblastoma (differentiated, undifferentiated)
CCL-11 mouse fibrosarcoma
Shin3 human ovarian carcinoma
PANC1 human pancreatic carcinoma
Primary murine bone marrow cells

Cells were cultivated using their respective optimized growth medium and passaged as described. Differentiation was induced in N18 cells by stepwise reduction of serum in the growth medium from 10% to 1.25%.

Bone marrow cells were extracted from hind legs of sacrificed mice and were kept in supplemented IMDM medium.

All cell types contacted with CyLoP-1 except differentiated N18 showed comparable uptake behaviour with vesicular as well as diffused cytosolic fluorescence distribution. Uptake into differentiated N18 neuroblastoma cells was strongly reduced especially into cells showing more neuronal morphology.

### EXAMPLE 6

### Determination of Concentrations of Fluorescently Labeled Peptides

Peptides were dissolved in MilliQ water to obtain a 10 mM solution by weight. For the determination of the exact peptide concentration, these stock solutions were diluted 1:100 in DMEM. The absorbance of the solutions was measured by multiplate reader (BMG Labtech, Germany) at 485 nm with ratiometric correction of turbidity at 690 nm. The concentrations of the stock solutions were calculated assuming ε_{carboxyfluorescein 485 nm}=81,000 l/(mol·cm). The concentrations of TAMRA labeled peptides was calculated assuming ε_{TAMRA 540 nm}=40,000 l/(mol·cm).

### EXAMPLE 7

### Uptake Assay

Internalization experiments on cells were performed in 96 well microplates by inoculation of NIH 3T3 fibroblasts (1×10⁴ cells/well). After 24 h, cells were incubated with 2.5 μM of different peptide solutions in DMEM for additional 18 h at 37° C. with 10% CO₂. Before washing, cells were incubated with Bisbenzimid 33342 (Hoechst 33342), a nuclear stain, in order to estimate the cell number. Cells were washed with Hanks' buffered saline (Biochrom AG, Germany) and extracellular fluorescence was quenched by incubating with cold trypan blue 0.05% (w/v) in phosphate-buffered saline (Biochrom AG, Germany) for 3 min followed by repeated washes with Hanks' buffered saline. Cell-related FITC fluorescence (Ex 485 nm/Em 530 nm) and cell number (Ex 346 nm/Em 460 nm) was evaluated in a multiplate reader. Experiments were run at least three times for each peptide with six replicates. Statistical analysis was performed by Student's t-test or ANOVA with Dunnett's post test. P values <0.05 were considered significant. The results are depicted in FIGS. 1 to 3.

Internalization of CyLoP-1 was compared to four known CPP (see table below). All peptides were synthesized and characterized in house as described in Examples 1 and 2. Cell uptake studies were done as described above.

| CPP | Sequence |
|---|---|
| Octaarginine (d-R8) | rrrrrrrr |
| d-Tat₄₉₋₅₇ (d-Tat) | rkkrrqrrr |
| d-Tat₅₇₋₄₉ (ri-Tat) | rrrqrrkkr |
| Penetratin (Antp) | RQIKIWFQNRRMKWKK |

K(FITC) or k(FITC) were coupled to the N-terminus of peptides.
Capitals denote L-amino acids, minuscules denote D-amino acids.

All four known CPP showed a significantly decreased internalization compared to CyLoP-1 (FIG. 7). Especially noteworthy is the predominantly vesicular appearance of fluorescence inside the cells for these CPP indicating endosomal entrapment and a lack of release into the cytosol.

### EXAMPLE 8

### Microscopy

The same cells were subjected to microscopic studies without fixation using a Zeiss Axiovert 200 M (Germany) microscope with a LD Plan NeoFluor 40× objective. The imaging conditions were kept constant for the observation of different samples. Cellular localization and distribution of the peptide was observed by irradiating with blue light (470/40 nm) and observing at 525/50 nm. The bright punctate and encapsulated appearing FITC fluorescence was categorized as vesicular uptake while diffused fluorescence appeared to be distributed in the entire cell with similar intensity. Manual observation of at least three experiments was compiled to conclude if the peptide shows diffused, vesicular or both types of uptake. Apart from FITC fluorescence, the nuclear labeling by Hoechst was observed at 460/50 nm and trypan blue fluorescence viewed at 645/75 nm. Also phase contrast images with DIC of the same area were made to observe if the cells maintain their normal morphology in the presence of peptides.

### EXAMPLE 9

### Time Course

Cells were seeded at a density of 1×10⁴ cells/well in 96 well microplates. After 48 h, cells were incubated with 2.5 μM of peptide solution in DMEM for different time points (30 min-18 h) at 37° C. Labeling with Hoechst, quenching and washings were performed as explained in detail in uptake assay. Fluorescence spectroscopy and microscopy was performed on the plate.

### EXAMPLE 10

### Effect of Temperature on Uptake

Cells were seeded at a density of 1×10⁴ cells/well in 96 well microplates. After 48 h, cells were incubated with 2.5 μM of different peptide solutions in DMEM for additional 4 h at 37° C. or 4° C. Labeling with Hoechst, quenching and washings were performed as explained in detail in uptake assay. Fluorescence spectroscopy and microscopy was performed on the plate.

### EXAMPLE 11

### Elucidating the Mechanism of Uptake

In order to understand the mechanism of internalization, cells were treated with a variety of inhibitors of uptake like wortmannin, methyl-β-cyclodextrin, NaN₃/2-deoxyglucose, chloroquine, etc. Cells were cultured at a density of 1×10⁴ cells/well in 96 well microplates. After 48 h, cells were pre-incubated with indicated inhibitors for 30 min followed by additional 4 h co-incubation with 2.5 μM of different peptide solutions in serum free DMEM at 37° C. or 4° C. Labeling with Hoechst, quenching and washings were performed as mentioned in detail in uptake assay. Fluorescence spectroscopy and microscopy was performed on the plate.

### EXAMPLE 12

### Peptide NMR

A potential participation of cysteines in the internalization activity of CyLoP-1 was further demonstrated by high resolution NMR studies on CyLoP-1 (with and without N-terminal lysine(FITC)). At pH 4.5 in water, the peptide was found to be unstructured and in a reduced state, whereas by increasing the pH to 6.5 a broadening of resonances was observed indicating aggregation under cellular uptake conditions.

Sample solutions were prepared at pH 4-4.5 and pH 6.5-7 in water. At pH 4.5 standard set of 2D homonuclear experiments (2D-ipCOSY, 2D-TOCSY, two 2D-NOESY spectra at different mixing times (100 and 200 ms)) and heteronuclear experiments (¹³C-HSQC and ¹⁵N-HSQC) were performed. The sample yielded well resolved spectra consistent with a monomeric peptide. Using these spectra it was possible to complete a full assignment of all non-exchangeable proton resonances, plus the carbon and nitrogen resonances. Low chemical shift dispersion, the lack of deviation of coupling constants from 7 Hz, and the amide protons showing either intra-residue contacts or contacts to the previous residue in the chain revealed the peptide to be unstructured.

Whereas at pH 6.5, broadening of lines, probably due to the formation of high molecular weight aggregates, makes it difficult to assign individual resonances. Thus, the presence or absence of disulfide bridges in this sample could not be deduced.

NMR experiments were carried out at 600 MHz on a Bruker Avance III spectrometer.

### REFERENCES

Altschul, S. F., Gish, W., Miller, W., Myers, E. W., Lipman, D. J., Basic local alignment search tool: J Mol Biol. 1990; 215(3):403-10.
Altschul, S. F. and Gish, W. Methods Enzymol. 1996, 266:460
Andreu, D., Albericio, F., Solé, N. A., Munson, M. C., Ferrer, M., Barany, G., in Peptide Synthesis Protocols, Methods Mol. Biol., Vol. 35 (Eds: M. W. Pennington, B. M. Dunn), Humana Press Inc., Totowa, N.J. 1994, pp. 91-169.
Ausubel, “Current Protocols in Molecular Biology”, Green Publishing Associates and Wiley Interscience, N.Y. (1989)
Barnes, M., Freudenberg, J., Thompson, S., Aronow, B., Pavlidis, P., Experimental comparison and cross-validation of the Affymetrix and Illumina gene expression analysis platforms: Nucleic Acids Res. 2005 Oct. 19; 33(18):5914-23. Print 2005.
Bebbington, C. R., G. Renner, S. Thomson, D. King, D. Abrams, and G. T. Yarranton. 1992. High-level expression of a recombinant antibody from myeloma cells using a glutamine synthetase gene as an amplifiable selectable marker. Biotechnology (N Y). 10:169-75.
Beltran, J. R. et al, Saxs study of structure and conformational changes of crotamine: Biophys. J. 47 (1985) 33-35.
Boni-Mitake, M. et al, Effects of ⁶⁰Co gamma radiation on crotamine: Braz J Med Biol Res, December 2001, Volume 34(12) 1531-1538.
Boni-Mitake, M. et al, Distribution of ¹²⁵I-labeled crotamine in mice tissues: Toxicon 48 (2006) 550-555.
Deshayes, S. et al, Cell-penetrating peptides: tools for intracellular delivery of therapeutics: Cell. Mol. Life Sci. 62 (2005) 1839-1849.
Casare, M. S. et al, Effects of 60Co radiation on the molecular structure of crotamine: Radiation Physics and Chemistry 71 (2004) 417-418.
Derossi, D., Joliot, A. H., Chassaing, G., Prochiantz, A., The third helix of the Antennapedia homeodomain translocates through biological membranes: J Biol Chem. 1994; 269(14):10444-50.
Egholm, M., Buchardt, O., Christensen, L., Behrens, C., Freier, S. M., Driver, D. A., Berg, R. H., Kim, S. K., Norden, B., Nielsen, P. E., PNA hybridizes to complementary oligonucleotides obeying the Watson-Crick hydrogen-bonding rules: Nature. 1993 Oct. 7; 365(6446):566-8.
Elmquist, A. et al, In vitro uptake and stability study of pVEC and its all-D analog: Biol Chem. 384 (2003) 387-93.
Fadel, V. et al, Automated NMR structure determination and disulfide bond identification of the myotoxin crotamine from Crotalus durissus terrificus: Toxicon 46 (2005) 759-767.
Fan, J. B., Hu, S. X., Craumer, W. C., Barker, D. L., BeadArray-based solutions for enabling the promise of pharmacogenomics. Biotechniques. 2005 October; 39(4):583-8.
Fernandez-Carneado, J. et al, Potential Peptide Carriers: Amphipathic Proline-Rich Peptides Derived from the N-Terminal Domain of γ-Zein: Angew. Chem. Int. Ed. 43 (2004) 1811-1814.
Fernandez-Carneado, J. et al, Fatty acyl moieties: improving Pro-rich peptide uptake inside HeLa cells: J. Peptide Res 65 (2005) 580-590.
Futaki, S. et al, Arginine-rich Peptides—An abundant source of membrane-permeable peptides having potential as carriers for intracellular protein delivery: J Biol Chem 276 (2001) 5836-5840.
Green, M., Loewenstein, P. M., Autonomous functional domains of chemically synthesized human immunodeficiency virus tat trans-activator protein. Cell. 1988 Dec. 23; 55(6):1179-88.
Gupta, B. et al, Intracellular delivery of large molecules and small particles by cell-penetrating proteins and peptides: Adv. Drug Delivery Rev. 57 (2005) 637-651.
Hampe, O. G. et al, Model studies of crotamine self-association: Braz J Med Biol Res 22 (1989) 17-24.
Hampe, O. G. et al, Polyacrylamide gel electrophoretic studies on the self association of crotamine: Characterization and molecular dimension of n-mer species: Electrophoresis 11 (1990) 415-478.
Hampe, O. G. et al, An in vivo method for estimating the concentration of Crotamine in solution: J. Venom. Anim. Toxins 3 (1997) 33-36.
Heckl, S. Sturzu, A., Regenbogen, M., Feil, G., Gharabaghi, A., Echner, H., A novel polyarginine containing Smac peptide conjugate that mediates cell death in tumor and healthy cells: Med Chem. 2008; 4(4):348-54.
Henikoff, S., Henikoff, J. G., Amino acid substitution matrices from protein blocks., Proc Natl Acad Sci USA. 1992 Nov. 15; 89(22):10915-9.
Higgins and Hames (Eds.) “Nucleic acid hybridization, a practical approach” IRL Press Oxford, Washington D.C., (1985)
Joliot, A., Pernelle, C., Deagostini-Bazin, H., Prochiantz, A., Antennapedia homeobox peptide regulates neural morphogenesis. Proc Natl Acad Sci USA. 1991 Mar. 1; 88(5):1864-8.
Kerkis, A. et al, Properties of Cell Penetrating Peptides (CPPs): IUBMB Life 58 (2006) 7-13.
Kerkis, A. et al, Crotamine is a novel cell-penetrating protein from the venom of rattlesnake Crotalus durissus terrificus: FASEB J 18 (2004) 1407-1409.
Lundberg, P. et al, A brief introduction to cell-penetrating peptides: J. Mol. Recognit. 16 (2003) 227-233.
Mae, M. and Langel, U., Cell-penetrating peptides as vectors for peptide, protein and oligonucleotide delivery: Curr Opin Pharmacol 6 (2006) 509-514.
Magzoub, M. et al, Cell-penetrating peptides: small from inception to application: Quart Rev Biophys 37 (2004) 147-195.
Mancin, A. C. et al, The analgesic activity of crotamine, a neurotoxin from Crotalus durissus terrificus (South American Rattle snake) venom: A biochemical and pharmacological study: Toxicon 36 (1998) 1927-1937.
Mascarenhas, Y. P. et al, Structure-function relationship for the highly toxic crotoxin from Crotalus durissus terrificus: Eur Biophys J 21 (1992) 199-205.
Matavel, A. C. S. et al, Tension generation and increase in voltage-activated Na⁺ current by crotamine: Eur J Pharmacol 348 (1998) 167-173.
Melikov, K. et al, Arginine-rich cell penetrating peptides: from endosomal uptake to nuclear delivery: Cell. Mol. Life Sci. 62 (2005) 2739-2749.
Michalsky, E. et al., Loops In Proteins (LIP)— a comprehensive loop database for homology modelling. Protein Eng. 16 (2003):979-985.
Mitchell, D. J. et al, Polyarginine enters cells more efficiently than other polycationic homopolymers: J Peptide Res 56 (2000) 318-325.
Murphy, G., M. I. Cockett, R. V. Ward, and A. J. Docherty. 1991. Matrix metalloproteinase degradation of elastin, type IV collagen and proteoglycan. A quantitative comparison of the activities of. 95 kDa and 72 kDa gelatinases, stromelysins-1 and -2 and punctuated metalloproteinase (PUMP). Biochem J. 277 (Pt 1):277-9.
Nascimento, F. D. et al, Crotamine mediates gene delivery into cells through the binding to heparan sulfate proteoglycans: J. Biol. Chem (2007).
Nicastro, G. et al, Solution structure of crotamine, a Nat channel affecting toxin from Crotalus durissus terrificus venom: Eur. J. Biochem. 270, 1969-1979 (2003).
Nielsen, P. E., Egholm, M., Berg, R. N., Buchardt, O., Sequence-selective recognition of DNA by strand displacement with a thymine-substituted polyimide. Science. 1991 Dec. 6; 254(5037):1497-500.
Novais, M. et al, 99 mTc radiolabeling of crotoxin as a tool for biodistribution studies: J. Radioanalyt. Nucl. Chem. 269 (2006) 591-595.
Pearson, W. R., Lipman, D. J., Improved tools for biological sequence comparison. Proc Natl Acad Sci USA. 1988 April; 85(8):2444-8.
Pooga, M., Soomets, U., Hällbrink, M., Valkna, A., Saar, K., Rezaei, K., Kahl, U., Hao, J. X., Xu, X. J., Wiesenfeld-Hallin, Z., Hökfelt, T., Bartfai, T., Langel, U., Cell penetrating PNA constructs regulate galanin receptor levels and modify pain transmission in vivo. Nat Biotechnol. 1998 September; 16(9):857-61.
Oguiura, N. et al, New view on crotamine, a small basic polypeptide myotoxin from South American rattlesnake venom: Toxicon 46 (2005) 363-370.
Owens, G. C., Chappell, S. A., Mauro, V. P., Edelman, G. M., Identification of two short internal ribosome entry sites selected from libraries of random oligonucleotides. Proc Natl Acad Sci USA. 2001 Feb. 13; 98(4):1471-6.
Rádis-Baptista, G. et al, Structure and chromosomal localization of the gene for crotamine, a toxin from the South American rattlesnake, Crotalus durissus terrificus: Toxicon 42 (2003) 747-752.
Rádis-Baptista, G., Oguiura, N., Hayashi, M. A., Camargo, M. E., Grego, K. F., Oliveira, E. B., Yamane, T., Nucleotide sequence of crotamine isoform precursors from a single South American rattlesnake (Crotalus durissus terrificus). Toxicon. 1999 July; 37(7):973-84.
Richard, J. P. et al, Cell-penetrating Peptides: a Reevaluation of the mechanism of cellular uptake: J Biol Chem 278 (2003) 585-590.
Rothbard, J. B. et al, Arginine-Rich Molecular Transporters for Drug Delivery: Role of Backbone Spacing in Cellular Uptake: J. Med. Chem. 45 (2002) 3612-3618.
Sambrook et al., “Molecular Cloning, A Laboratory Manual”; CSH Press, Cold Spring Harbor, 1989
Sarin, V. K. et al, Quantitative monitoring of solid-phase peptide synthesis by the ninhydrin reaction: Anal. Biochem. 117 (1981) 147-157.
Sauer, M. G., Ericson, M. E., Weigel, B. J., Herron, M. J., Panoskaltsis-Mortari, A. Kren, B. T., Levine, B. L., Serody, J. S., June, C. H., Taylor, P. A., Blazar, B. R. A novel system for simultaneous in vivo tracking and biological assessment of leukemia cells and ex vivo generated leukemia-reactive cytotoxic T cells. Cancer Res. 2004, 64:3914-3921.
Shen, R., Fan, J. B., Campbell, D., Chang, W., Chen, J., Doucet, D., Yeakley, J., Bibikova, M., Wickham Garcia, E., McBride, C., Steemers, F., Garcia, F., Kermani, B. G., Gunderson, K., Oliphant, A., High-throughput SNP genotyping on universal bead arrays. Mutat Res. 2005 Jun. 3; 573(1-2):70-82. Review.
Smith, T. F., Waterman, M. S., Identification of common molecular subsequences. J Mol Biol. 1981 Mar. 25; 147(1):195-7.
Soomets, U., Lindgren, M., Gallet, X., Hallbrink, M., Elmquist, A., Balaspiri, L., Zorko, M., Pooga, M., Brasseur, R., Langel, U., Deletion analogues of transportan. Biochim Biophys Acta. 2000 Jul. 31; 1467(1):165-76.
Steemers, F. J., Gunderson, K. L., Illumine, Inc. Pharmacogenomics. 2005 October; 6(7):777-82.
Su, W., Mishra, R., Pfeuffer, J., Wiesmüller, K. H., Ugurbil, K., Engelmann, Synthesis and cellular uptake of a MR contrast agent coupled to an antisense peptide nucleic acid-cell-penetrating peptide conjugate. Contrast Media and Molecular Imaging 2 (2007) 42-49
Takeuchi, T. et al, Direct and Rapid Cytosolic Delivery Using Cell-Penetrating Peptides Mediated by Pyrenebutyrate: ACS Chem. Biol. 1 (2006) 299-303.
Thompson, J. D., Higgins, D. G., Gibson, T. J., CLUSTAL W: improving the sensitivity of progressive multiple sequence alignment through sequence weighting, position-specific gap penalties and weight matrix choice. Nucleic Acids Res. 1994 Nov. 11; 22(22):4673-80.
Toyama, M. H. et al, Biophysical, histopathological and pharmacological characterization of crotamine isoforms F22 and F32: Toxicon 41 (2003) 493-500.
Toyama, M. H. et al, Biochemical characterization of two crotamine isoforms isolated by a single step RP-HPLC from Crotalus durissus terrificus (South American rattlesnake) venom and their action on insulin secretion by pancreatic islets: Biochim Biophy Acta 1474 (2000) 56-60.
Tréhin, R. and Merkle, H. P., Chances and pitfalls of cell penetrating peptides for cellular drug delivery: Eur J Pharm Biopharm 58 (2004) 209-223. Proc Natl Acad Sci USA. 2000 Nov. 21; 97(24):13003-8.
Wacker, B. K., Alford, S. K., Scott, E. A., Thakur, M. D., Longmore, G. D., Elbert, D. L., Endothelial cell migration on RGD-peptide-containing PEG hydrogels in the presence of sphingosine 1-phosphate: Biophys J. 2008; 94(1):273-85.
Wender P A, Mitchell D J, Pattabiraman K, Pelkey E T, Steinman L, Rothbard J B., Zorko, M. et al, Cell-penetrating peptides: mechanism and kinetics of cargo delivery: Adv Drug Delivery Rev 57 (2005) 529-545.

1. A nucleic acid molecule encoding a peptide capable of being internalized into a cell, wherein said nucleic acid molecule is selected from the group consisting of:
(a) a nucleic acid molecule encoding a peptide having the amino acid sequence of SEQ ID NO: 2;
(b) a nucleic acid molecule having the DNA sequence of SEQ ID NO: 1, wherein T is U if the nucleic acid molecule is RNA; and
(c) a nucleic acid molecule encoding a peptide having at least 80% sequence identity with that of SEQ ID NO: 2, wherein at least at two positions selected from the group consisting of positions 1, 7 and 8 of SEQ ID NO: 2 a cysteine is present and wherein at least at four positions selected from the groups consisting of position 2, 4, 6, 9 and 10 of SEQ ID NO: 2 a positively charged amino acid is present.

2. The nucleic acid molecule of claim 1 wherein the peptide further comprises a linker.

3. The nucleic acid of claim 2, wherein said linker is located at the N-terminus of said peptide.

4. The nucleic acid molecule of claim 2, wherein the linker is a lysine.

5. A vector comprising the nucleic acid molecule of claim 1.

6. A non-human host transfected or transformed with the vector of claim 5.

7. The non-human host of claim 6 which is a cell.

8. A method of producing a peptide comprising culturing the non-human host cell of claim 7 under suitable conditions and isolating the peptide produced.

9. A peptide produced by the method of claim 8.

10. The peptide of claim 9, wherein the peptide is modified by acylation, amidation, esterification, glycosylation, phosphorylation, biotinylation, PEGylation, or coupling of farnesyl.

11. The peptide of claim 9, wherein a disulfide bond is present between the cysteines at positions 1 and 7 or 7 and 8 of SEQ ID NO: 2.

12. The peptide of claim 11, wherein the peptide is modified by acylation, amidation, esterification, glycosylation, phosphorylation, biotinylation, PEGylation, or coupling of farnesyl.

13. The peptide of claim 9, wherein the C-terminus of the peptide is modified.

14. The peptide of claim 13, wherein the peptide is modified by acylation, amidation, esterification, glycosylation, phosphorylation, biotinylation, PEGylation, or coupling of farnesyl.

15. A fusion molecule comprising the peptide encoded by the nucleic acid molecule of claim 1.

16. The fusion molecule of claim 15, wherein the peptide is fused to a nucleic acid, a protein or peptide, an aptamer, a small molecule, a nanoparticle or nanocarrier or a contrast agent.

17. The fusion molecule of claim 16, wherein the peptide is fused to FITC, TAMRA, Gd-DOTA-, Gd-DTPA-⁶⁴Cu-DOTA-, ⁶⁸Ga-DOTA, an siRNA, an antisense oligonucleotide or a nucleic acid molecule encoding one or more reporter genes.

18. A pharmaceutical composition comprising the peptide encoded by the nucleic acid molecule of claim 1 or the fusion molecule of claim 15.

19. The pharmaceutical composition of claim 18 further comprising a pharmaceutically acceptable carrier, excipient and/or diluent.

20. The pharmaceutical composition of claim 18 which is a vaccine.

21. A diagnostic composition comprising at least one of
(a) the nucleic acid molecule of claim 1,
(b) the vector of claim 5,
(c) a peptide encoded by the nucleic acid molecule of claim 1, or
(d) the fusion molecule of claim 15.

22. A method of detecting the cellular internalization of a molecule comprising:
(a) contacting a cell with a molecule selected from the group consisting of the nucleic acid molecule of claim 1, a peptide encoded by the nucleic acid molecule of claim 1, and the fusion molecule of claim 15; and
(b) detecting the internalization of the molecule.

23. A method of treating or preventing a condition in a subject comprising administering the a peptide encoded by the nucleic acid molecule of claim 1, or the fusion molecule of claim 15 to a subject in need thereof.

24. The method of claim 23 wherein the condition is selected from the group consisting of cancer, enzyme deficiency diseases, infarcts, cerebral ischemia, diabetes, inflammatory diseases, infections, autoimmune diseases, diseases with amyloid-like fibrils and forms of myopathy.

25. The method of claim 24 wherein the infection is selected from the group consisting of bacterial viral and fungal.

26. The method of claim 24 wherein the autoimmune disease is selected from the group consisting of systemic lupus erythematosus (SLE) and rheumatoid arthritis.

27. The method of claim 24 wherein the disease with amyloid-like fibrils is selected from the group consisting of Alzheimer's disease (AD) and Parkinson's disease (PD).

28. A kit comprising at least one of
(a) the nucleic acid molecule of claim 1,
(b) the vector of claim 5,
(c) the non-human host cell of claim 7,
(d) the peptide encoded by the nucleic acid molecule of claim 1, or
(e) the fusion molecule of claim 15.
